SEQUENCE LISTING (10 sequences)

SEQ ID NO: 1 - DNA, 45 bp, Artificial Sequence
  Synthetic construct  KRAS codon 12 WT oligonucleotide
cggcaaacggcatcaaacggcataaacttgtggtagttggagctg

SEQ ID NO: 2 - DNA, 45 bp, Artificial Sequence
  Synthetic construct  KRAS codon 12 mutant (G to R) oligonucleotide
cggcaaacggcatcaaacggcataaacttgtggtagttggagctc

SEQ ID NO: 3 - DNA, 44 bp, Artificial Sequence
  Synthetic construct  p53 codon 175 mutant (R to H) oligonucleotide
cggcaaacggcatcaaacggccacatgacggaggttgtgaggca

SEQ ID NO: 4 - DNA, 44 bp, Artificial Sequence
  Synthetic construct  p53 codon 248 mutant (R to Q) oligonucleotide
cggcaaacggcatcaaacggctcctgcatgggcggcatgaacca

SEQ ID NO: 5 - DNA, 50 bp, Artificial Sequence
  Synthetic construct  Positive control oligonucleotide sequence, linked to TEG-biotin at 3′ end.
catcacacaacatcacacaacatcacgtatataaaacggaacgtagaagg

SEQ ID NO: 6 - DNA, 50 bp, Artificial Sequence; identical to SEQ ID NO: 5
  Synthetic construct  Non-specific oligonucleotide sequence.

SEQ ID NO: 7 - DNA, 66 bp, Artificial Sequence
  Synthetic construct  KRAS codon 12 WT target oligonucleotide
ttagctgtatcgtcaaggcactcttgcctacgccaccagctccaactaccacaagtttatattcag

SEQ ID NO: 8 - DNA, 68 bp, Artificial Sequence
  Synthetic construct  KRAS codon 12 mutant (G to R) target oligonucleotide
aattagctgtatcgtcaaggcactcttgcctacgccacgagctccaactaccacaagtttatattcag

SEQ ID NO: 9 - DNA, 66 bp, Artificial Sequence
  Synthetic construct  p53 codon 175 mutant (R to H) target oligonucleotide
accatcgctatctgagcagcgctcatggtgggggcagtgcctcacaacctccgtcatgtgctgtga

SEQ ID NO: 10 - DNA, 66 bp, Artificial Sequence
  Synthetic construct  p53 codon 248 (R to Q) mutant target oligonucleotide
ggagtcttccagtgtgatgatggtgaggatgggcctctggttcatgccgcccatgcaggaactgtt

(19) United States
(12) Patent Application Publication
(10) Pub. No.: US 2009/0137405 A1
(43) Pub. Date: May 28, 2009

(54) DETECTION OF NUCLEIC ACID BIOMARKERS USING POLYMERIZATION-BASED AMPLIFICATION

(76) Inventors: CHRISTOPHER BOWMAN, BOULDER, CO (US); RYAN HANSEN, GOLDEN, CO (US); LEAH JOHNSON, WESTMINSTER, CO (US); HADLEY SIKES, PASADENA, CA (US); HEATHER AVENS, LAFAYETTE, CO (US)

Correspondence Address: GREENLEE WINNER AND SULLIVAN P C, 4875 PEARL EAST CIRCLE, SUITE 200, BOULDER, CO, 80301, US

(21) Appl. No.: 12/272,539

(22) Filed: Nov. 17, 2008

Related U.S. Application Data
(60) Provisional application No. 60/988,563, filed on Nov. 16, 2007.

(51) Int. Cl. C40B 20/02; C12Q 1/68

(52) U.S. Cl. 506/3; 435/6

(57) ABSTRACT

The invention provides methods for highly-specific detection of hybridization of single stranded nucleic acids. The invention also provides methods for target identification which rely on this highly-specific hybridization detection. Targets suitable for detection include, but are not limited to, nucleic acid biomarkers. The methods of the invention can employ an on-chip, DNA polymerase-dependent labeling scheme termed primer extension (PEX) to couple biotinylated deoxyribonucleotide triphosphate (dNTP) molecules to nucleic acid hybrids bound to a solid substrate, allowing for subsequent recognition by biotin-binding-protein-labeled photoinitiators. Surface-initiated polymerization from these surface bound photoinitiators can lead to the formation of macroscale amounts of polymeric material, thereby amplifying the signal from the initial molecular recognition event.

### CROSS-REFERENCE TO RELATED APPLICATIONS

This application claims the benefit of U.S. Provisional Application No. 60/988,563 filed Nov. 16, 2007, which is hereby incorporated by reference to the extent not inconsistent with the disclosure herein.

### ACKNOWLEDGEMENT OF GOVERNMENT SUPPORT

This invention was made at least in part with support from the National Science Foundation under grant number SGER 0442047, from the National Institutes of Health under grant number R41 AI060057 and 1R21 CA 127884, and the Human Genome Research Institute (NSRA F32-HG003100). The United States Government has certain rights in this invention.

### BACKGROUND OF THE INVENTION

A variety of methods exist for detection of molecular recognition events. Detection of molecular recognition events such as DNA hybridization becomes increasingly difficult as the number of recognition events to be detected decreases. Of particular interest are molecular recognition events between a target and a probe.

One approach to the problem is to increase the number of recognition events taking place. For example, polymerase chain reaction (PCR) increases the number of copies of DNA or RNA to be detected. Other molecular biology techniques which increase the number of copies of DNA or RNA to be detected include reverse transcription polymerase chain reaction (RT-PCR), strand displacement amplification, and Eberwine linear amplification.

Another approach is to amplify the signal due to each molecular recognition event. For example, DNA detection methods based on oligonucleotide-modified particles have been reported (U.S. Pat. Nos. 6,740,491, 6,777,186, 6,773,884, 6,767,702, 6,759,199, 6,750,016, 6,730,269, 6,720,411, 6,720,147, 6,709,825, 6,682,895, 6,673,548, 6,667,122, 6,645,721, 6,610,491, 6,582,921, 6,506,564, 6,495,324, 6,417,340 and 6,361,944 and Park, S.-J. et al, 2002, Science, 295, 5559, 1503-1506). U.S. Pat. No. 6,602,669 relates to silver staining nanoparticles.

DNA detection methods based on branched DNA have also been reported (U.S. Pat. Nos. 5,681,702, 5,597,909, 5,580,731, 5,359,100, 5,124,246, 5,545,730, 5,594,117, 5,571,670, 5,594,118, 5,681,697, 5,591,584, 5,571,670, 5,624,802, 5,635,352, and 5,591,584. The branched DNA assay is a solution phase assay that involves a number of probe oligonucleotides that bind to multiple sites on the target viral RNA. Detection is possible because each hybridization event is accompanied by the binding of a fluorophore (Kern, D., Collins, M., Fultz, T., Detmer, J., Hamren, S., Peterkin, J., Sheridan, P., Urdea, M., White, R., Yeghiazarian, T., Todd, J. (1996) “An Enhanced-sensitivity Branched-DNA Assay for Quantification of Human Immunodeficiency Virus Type 1 RNA in Plasma” Journal of Clinical Microbiology 34:3196-3203). The synthetic effort required for this assay is relatively large: multiple probes are designed for each RNA of interest, and the assay depends on the binding of these probes to multiple preamplifier and amplifier molecules that also must be designed and synthesized.

Dendrimer-based DNA detection methods have also been reported (U.S. Pat. Nos. 5,710,264, 5,175,270, 5,487,973, 5,484,904 and Stears, R. et al., 2000, Physiol. Genomics 3: 93-99). Dendrimers are complexes of partially double-stranded oligonucleotides, which form stable, spherical structures with a determined number of free ends. Specificity of the dendrimer detection is accomplished through specific binding of a capture oligonucleotide on a free arm of the dendrimer. Other arms of the dendrimer are labeled for detection. This method does not require enzymes and can produce amplification of 300-400.

Tyramide signal amplification is reported in U.S. Pat. Nos. 6,593,100 and 6,372,937.

Rolling circle amplification has been described in the scientific literature (Baner et al. (1998) Nuc. Acids Res. 26:5073-5078; Barany, F. (1991) Proc. Natl. Acad. Sci. USA 88:189-193; Lizardi et al. (1998), Nat. Genet. 19:225-232; Zhang et al., Gene 211:277 (1998); and Daubendiek et al., Nature Biotech. 15:273 (1997)). Rolling circle amplification is capable of detecting as few as 150 molecules bound to a microarray (Nallur, G., Luo, C., Fang, L., Cooley, S., Dave, V., Lambert, J., Kukanskis, K., Kingsmore, S., Lasken, R., Schweitzer, B. (2001) “Signal Amplification by Rolling Circle Amplification on DNA Microarrays” Nucleic Acids Research 29:E118).

Ligase chain reaction is reported in U.S. Pat. Nos. 5,185,243 and 5,573,907.

Cycling probe technology is reported in U.S. Pat. Nos. 5,011,769, 5,403,711, 5,660,988, and 4,876,187.

Microfabricated disposable DNA sensors based on enzymatic amplification electrochemical detection was reported by Xu et al. (Xu et al., 2001, Electoanalysis, 13(10), 882-887).

Polymerization-based amplification has also been reported. U.S. Pat. No. 7,354,706 to Rowlen et al. reports use of photopolymerization for amplification and detection of a molecular recognition event. WO/2007/095464 to Kuck reports signal amplification of biorecognition events using photopolymerization in the presence of air. Sikes et al. (H. D. Sikes et al., Nat. Mater. 7 (2008) 52-56) reported a visual detection method utilizing photopolymerization for signal amplification on a commercially available biodetection platform (Jenison et al., Nature Biotechnology 2001, 19, 62-65; Jenison, R et al., Clinical Chemistry 2001, 47, 1894-1900). A more recent approach to signal amplification involves polymerization-based amplification (PBA) systems capable of direct visualization and detection of minute levels of biotinylated biomolecules present on glass slides (R. Hansen et al., Biomacromolecules 9 (2008) 355-362; R. R. Hansen, et al., Anal. Bioanal. Chem. 392 (2008) 167-175). These systems employ steptavidin (SA) labeled photoinitiators capable of both recognizing surface-bound biotin and eliciting the formation of macroscale polymeric material upon surface-initiated polymerization reactions.

Surface initiated polymerization from surface confined initiators has also been reported. Biesalski et al. report poly(methyl methacrylate) brushes grown in situ by free radical polymerization from an azo-initiator monolayer covalently bound to the surface (Biesalski, M. et al., (1999), J. Chem. Phys., 111(15), 7029). Surface initiated polymerization for amplification of patterned self-assembled monolayers by surface-initiated ring opening polymerization (Husemann, M. et al., Agnewandte Chemie Int. Ed. (1999), 38(5) 647-649) and atom transfer radical polymerization (Shah, R. R. et al., (2000), Macromolecules, 33, 597-605) has been also reported.

DNA microarrays, or biochips, represent promising technology for accurate and relatively rapid pathogen identification (Wang, D., Coscoy, L., Zylberberg, M., Avila, P. C., Boushey, H. A., Ganem, D., DeRisi, J. L. (2002) “Microarray Based Detection and Genotyping of Viral Pathogens,” PNAS, 99(24), 15687-15692). Anthony et al. recently demonstrated rapid identification of 10 different bacteria in blood cultures using a BioChip (Anthony, R. M., Brown, T. J., French, G. L. (2000) “Rapid Diagnosis of Bacteremia by Universal Amplification of 23S Ribosomal DNA Followed by Hybridization to an Oligonucleotide Array” Journal of Clinical Microbiology 38:781-788). The microarray assay was conducted in ˜4 hrs. The approach utilized universal primers for PCR amplification of the variable region of bacterial 23s ribosomal DNA, and a 3×10 array of 30 unique capture sequences. This work demonstrates an important aspect of BioChip platforms—the capability to screen for multiple pathogens simultaneously. DeRisi and co-workers demonstrated a “virus chip” that contained sequences for hundreds of viruses, including many that cause respiratory illness (Wang et al., 2002). This chip proved useful in identifying the corona virus associated with SARS (Risberg, E. (2003) “Gene Chip Helps Identify Cause of Mystery Illness,” USA Today (Jun. 18, 2003)). Evans and co-workers have demonstrated that a DNA microarray could be used for typing and sub-typing human influenza A and B viruses (Li, J., Chen, S., & Evans, D. H. (2001) “Typing and Subtyping Influenza Virus Using DNA Microarrays and Multiplex Reverse Transcriptase PCR” Journal of Clinical Microbiology 39:696-704). In both the DeRisi and Evans work PCR technology was used to amplify the genetic material for capture and relatively expensive fluorescent labels (˜$50 in labels per chip) were used to generate signals from positive spots. Townsend et al. report experimental evaluation of a FluChip diagnostic microarray for influenza virus surveillance (Townsend, M. et al., J. Clinical Microbiology, August 2006, 44(8), 2863-2871). Dawson et al. report DNA microarrays that target the matrix gene segment of influenza A (MChip) (Dawson, E. et al., October 2006, Anal. Chem., 78(22), 7610-7615; Dawson, E. et al, November 2006, Anal. Chem., 79 (1), 378-384, 2007).

There remains a need in the art for relatively inexpensive labeling and signal amplification methods for molecular recognition events, especially those useful in combination with DNA microarrays.

### SUMMARY OF THE INVENTION

In one aspect, the invention provides methods to detect hybridization between nucleic acids based on amplification of the signal due to each hybridization event. In another aspect, the invention provides methods for detection of a target nucleic acid based on amplification of the signal due to hybridization between the target nucleic acid and a probe molecule comprising a nucleic acid. In an embodiment, the probe molecule is part of an array of probe molecules attached to a solid substrate. Polymerization-based amplification (PBA) is used in both these aspects of the invention. In an embodiment, the methods of the invention can be used to detect nucleic acid biomarkers such as biomarkers for cancer or infectious disease. The ability to detect nucleic acid biomarkers with adequate sensitivity, mutation selectivity, and high-throughput capability offers the potential to provide advanced detection, evaluate tumor burden or viral and microbial load, predict personalized therapies, and monitor therapeutic efficacies (4-10).

In an embodiment, the invention provides a method for highly-specific DNA hybridization detection through the implementation of an on-chip, polymerase-dependent labeling scheme termed primer extension (PEX) (11). In an embodiment, this approach effectively couples biotinylated deoxyribonucleotide triphosphate molecules (dNTPs) to DNA hybrids bound to a solid substrate, allowing for subsequent recognition by streptavidin-labeled (SA-labeled) photoinitiators. Surface-initiated polymerization from these surface bound initiators can lead to the formation of macroscale amounts of polymeric material.

Combination of PEX labeling with a polymerization-based amplification method permits the rapid labeling of multiple hybridization sites for simultaneous, multiplexed amplification and detection using a single polymerization initiator reagent mixture. Further, the tremendous discriminatory potential of DNA polymerase enzymes enables high specificity in labeling, presenting advantages in SNP typing or point mutation detection applications (11-14) and potentially allows for allele-specific polymerization-based signal amplification, as illustrated in FIG. 1.

FIGS. 1A-1D provide a schematic of ideal polymerization-based amplification for detection of nucleic acid hybridization at single-base specificity. FIGS. 1A-D show different stages in the process at two probe sites. The probe at site A (10a) has a binding region (14a) which is perfectly complementary to a portion (24a) of the target molecule (20a). The probe at site B (10b) has a binding region (14b) which is not perfectly complementary to the corresponding portion (24b) of the target molecule (20b). Both probes are bound to the solid substrate (30) and include a spacer portion (16a, 16b).

FIG. 1A illustrates duplexes formed at the two probe sites after hybridization of the same target molecule sequence to the two different probe sequences. At Site A, the binding region (14a) of the probe is perfectly complementary to the binding region of the target (24a). At Site B, there are mismatched bases at the 3′ terminus of the probe molecule (mismatch region 12b). At both Sites A and B, an extension region (22a, 22b) is formed at the 5′ end of the target; this is a non-binding region at this stage of the process. In addition, an overhang region of mismatched bases (26a, 26b) is formed at the 3′ end of the target at both sites; this mismatch region “caps” the 3′ end of the target sequence and prevents extension from this terminus.

FIG. 1B illustrates the two probe sites after the primer extension step. At Site A, where perfectly complementary binding occurs between the binding region of the probe and the target, biotin labeled dNTPs (shown here as biotin-dATP) have been incorporated into the extension product (11a). At site B where non-specific hybridization occurs at the 3′ end of the probe binding sequence, the mismatch prevents formation of an extension product.

FIG. 1C illustrates the two probe sites after attachment of a photoinitiator label via interaction of a biotin-binding protein in the photoinitiator label with biotin in the extension product. In FIG. 1C, the photoinitiator label is shown as a streptavidin modified eosin isothiocyanate component (SA-EITC). No photoinitiators are attached at site B, demonstrating preferential photoinitiator labeling at sites where perfectly complementary binding occurs (site A).

FIG. 1D schematically illustrates locations of polymer growth during the polymerization based amplification stage. During polymerization based amplification, the presence of photoinitiators at site A can allow polymer growth at this site. However, no polymer growth will occur from site B since it lacks photoinitiators. This allows use of polymerization-based amplification for detection of nucleic acid hybridization at high specificity.

In an embodiment, hybridization occurs between a single stranded target nucleic acid and a probe comprising a single stranded nucleic acid, the 5′ end of the probe being bound to a solid substrate. The target and probe are two species of interest which undergo molecular recognition. In an embodiment, at least some characteristics of the probe and target are known. In an embodiment, the target comprises an oligonucleotide whose sequence is known or partially known. In an embodiment, the probe comprises an oligonucleotide whose sequence is known or partially known. In other embodiment, the sequence of the probe may not be known, but it is known to be complementary to a possible target species. Typically, the probe will be selected so that it is capable of selected recognition with the known or suspected identity of the target. In some cases a single probe can be used to detect the presence of a target. In other cases more than one probe will be necessary to detect the presence of or identify a target.

In an embodiment, the probe nucleic acid comprises a first region (probe binding region) and a second region (spacer region). The 3′ terminus of the probe binding region is at the 3′ terminus of the probe nucleic acid. In an embodiment, the length of the binding region of the probe is selected so that it is less than the length of the known or suspected target. In an embodiment, the binding region of the probe is selected to be the same length as and at least partially complementary to a portion of the selected target to be detected (the target binding region). In an embodiment, the binding region of the probe is selected to be perfectly complementary to the target at the 3′ terminus of the probe binding region. In another embodiment, the binding region of the probe is sufficiently complementary to the target to produce a stable hybrid.

In an embodiment, the binding region of the target is located at an intermediate position in the target (not located at either the 3′ or the 5′ end of the target molecule). Selection of a target binding region located away from the 5′ end of the molecule ensures that there will be a portion of the target located between the 5′ end of the target binding region and the 5′ end of the molecule (the extension region); this extension region later provides a template for polymerase assisted extension. Selection of a target binding region located away from the 3′ end of the target molecule provides an overhang from the 3′ end of the target binding region to the 3′ end of the target molecule (end cap region). To prevent polymerase assisted extension at the 3′ end of the target, the spacer region of the probe is selected so that is not complementary to the overhang/end cap region. In 3′ to 5′ order, the target nucleic acid may be said to comprise a first region (end cap region), a second region (binding region), and a third region (extension region).

In an embodiment, the duplex formed by successful hybridization of the target and probe is incubated in the presence of a DNA polymerase and a nucleotide triphosphate mixture, the nucleotide triphosphate mixture comprising at least one biotin-labeled nucleotide. In an embodiment, the nucleotide triphosphate mixture comprises a plurality of biotin-labeled and unlabeled dNTP molecules. In an embodiment, the nucleotide triphosphate mixture comprises a plurality of biotin-labeled dNTP molecules. The incubation takes place under conditions sufficient to permit extension of the 3′ terminus of the probe in a polymerase-mediated, template-dependent primer extension reaction for hybrids with sufficiently complementary sequences. The extension portion of the target acts as the template for formation of the extension product. In an embodiment, the concentration of biotin-labeled nucleotides triphosphates molecules is sufficiently high to allow the incorporation of at least one biotin-labeled nucleotide into the extension product. A biotin-labeled target probe complex is formed as a result of the primer extension reaction, where the biotin-labeled target-probe complex includes the original target probe duplex and the biotin-containing extension product.

In an embodiment, following primer extension the target-probe complex is labeled with a photoinitiator label which comprises a photoinitiator and a biotin-binding protein. Photoinitiators that are useful in the invention include those that can be activated with light and initiate polymerization of the polymer precursor. In an embodiment, the photoinitiator is consumed in the reaction and is not a photocatalyst. In an embodiment, the photoinitiator is water soluble. The photoinitiator is selected to be compatible with the wavelengths of light supplied.

In an embodiment, the photoinitiator is part of a two-part photoinitiator system, comprising a photoinitiator and a co-initiator. In an embodiment, the photoinitiator interacts with the co-initiator to generate free-radicals upon exposure to a source of visible light. The free-radicals can then be used to initiate polymerization of monomers via a chain-growth mechanism. The concept of amplification is inherent in chain-growth polymerization reactions due to the large number of propagation steps that result from a single initiation event. In an embodiment, the amplification factor can be up to 10⁶ or 10⁷. In another embodiment, the detectable response can be generated from as low as 10⁴ molecular recognition events.

In an embodiment, the photoinitiator is activated by visible light. Use of visible light sources for photoinitiation has the attractive characteristic of requiring only a low power, inexpensive and mild excitation source. Further, use of visible light has the added advantage of eliminating the unwanted bulk polymerization often observed when using UV light. The use of visible light, rather than UV light, for photoinitiation can also expand the range of suitable monomer formulations. In an embodiment, the monomer formulation contains high concentrations of bi-functional monomers that form thick, highly crosslinked polymer that remains stable on the surface with rinsing. Formation of a surface-stable hydrogel allows characterization of the amplification process with film thickness and spectroscopic measurements. Finally, visible light can enable more efficient amplification due to its higher penetration capability in UV absorbent monomer formulations containing fluorescent monomers or on UV absorbent surfaces characteristic of glass biochips containing surface-bound biomolecules.

In an embodiment, the invention provides a method for amplifying a molecular recognition interaction between a target and a probe, said method comprising the steps of:
a. providing a target single stranded nucleic acid (target), the target comprising in 3′ to 5′ order a first region (end cap region), a second region (binding region), and a third region (extension region);
b. providing a probe comprising a single stranded nucleic acid, said nucleic acid comprising in 3′ to 5′ order a first region (binding region) and a second region (spacer region), the 5′ end of the second region being bound to a solid substrate, wherein the first region of the target is of shorter length than and is not complementary to the second region of the probe, and the second region of the target is of equal length to and is at least partially complementary to the first region of the probe;
c. contacting the target with the probe under conditions effective to form a duplex between the probe and the target by hybridization of the first region of the probe to the second region of the target;
d. removing target not complexed with the probe;
e. incubating the duplex of step (c) in the presence of a DNA polymerase and a nucleotide triphosphate mixture, the nucleotide triphosphate mixture comprising at least one biotin-labeled nucleotide triphosphate, the incubation being under conditions sufficient to permit extension of the 3′ terminus of the first region of the probe in a polymerase-mediated, template-dependent primer extension reaction and incorporation of the biotin-labeled nucleotide into the extension product, thereby forming a biotin-labeled target probe complex;
f. contacting the biotin-labeled target-probe complex with a photoinitiator label comprising a photoinitiator and a biotin binding protein under conditions effective to attach the photoinitiator label to the target-probe complex, thereby forming a photoinitiator-labeled target-probe complex;
g. removing photoinitiator label not attached to the target-probe complex;
h. contacting the photoinitiator-labeled target-probe complex with a polymer precursor solution;
i. exposing the photoinitiator-labeled target-probe complex and the polymer precursor solution to light, thereby forming a polymer; and
j. detecting the polymer formed in step (i), thereby detecting an amplified target-probe interaction

In another embodiment, following primer extension the biotin-labeled target-probe complex is labeled with a biotin-binding protein through interaction between the biotin and the biotin-binding protein. This biotin-binding protein labeled target-probe complex may then be labeled with a photoinitiator label which comprises a photoinitiator and a biotin via interaction between the biotin and the biotin-binding protein.

In another embodiment, the invention provides a method for amplifying a molecular recognition interaction between a target and a probe, said method comprising the steps of:
a. providing a target single stranded nucleic acid (target), the target comprising in 3′ to 5′ order a first region (end cap region), a second region (binding region), and a third region (extension region);
b. providing a probe comprising a single stranded nucleic acid, said nucleic acid comprising in 3′ to 5′ order a first region (binding region) and a second region (spacer region), the 5′ end of the second region being bound to a solid substrate, wherein the first region of the target is of shorter length than and is not complementary to the second region of the probe, and the second region of the target is of equal length to and is at least partially complementary to the first region of the probe;
c. contacting the target with the probe under conditions effective to form a duplex between the probe and the target by hybridization of the first region of the probe to the second region of the target;
d. removing target not complexed with the probe;
e. incubating the duplex of step (c) in the presence of a DNA polymerase and a nucleotide triphosphate mixture, the nucleotide triphosphate mixture comprising at least one biotin-labeled nucleotide triphosphate, the incubation being under conditions sufficient to permit extension of the 3′ terminus of the first region of the probe in a polymerase-mediated, template-dependent primer extension reaction and incorporation of the biotin-labeled nucleotide into the extension product, thereby forming a biotin-labeled target probe complex;
f. contacting the biotin-labeled target-probe complex with a biotin-binding protein, thereby forming a biotin-binding-protein-labeled target probe complex;
g. contacting the biotin-binding-protein-labeled target-probe complex with a photoinitiator label comprising a photoinitiator and biotin under conditions effective to attach the photoinitiator label to the target-probe complex, thereby forming a photoinitiator-labeled target-probe complex;
h. removing photoinitiator label not attached to the target-probe complex;
i. contacting the photoinitiator-labeled target-probe complex with a polymer precursor solution;
j. exposing the photoinitiator-labeled target-probe complex and the polymer precursor solution to light, thereby forming a polymer; and
k. detecting the polymer formed in step (j), thereby detecting an amplified target-probe interaction

In another embodiment, the invention provides a method for identifying a target, the method comprising the steps of providing a probe array comprising a plurality of different probes, wherein the probes are attached to a solid substrate at known locations; and amplifying the molecular recognition interaction between the target and the probes by the methods of the invention, wherein the target is contacted with the probe by contacting the probe array with the target and the location of the polymer formed indicates the probe which forms a target-probe complex with the target, thereby identifying the target

### BRIEF DESCRIPTION OF THE FIGURES

FIGS. 1A-1D. Schematic of polymerization-based amplification for detection of nucleic acid hybridization at single-base specificity.

FIGS. 2A-2D. a) Array layout of biochip used for detection of p53 targets. b-d) Digital camera images of biochips for the detection of solutions of p53 target(s) b) 50 nM p53_{—}175 target in serum c) 50 nM p53_{—}248 target in serum d) 50 nM p53_{—}175 and 50 nM p53_{—}248 in serum.

FIGS. 3A-3B. a) Profilometry height profiles across five replicate spots generated from decreasing amounts of target concentrations in buffer after PEX labeling, SA-EITC functionalization, and polymerization-based amplification (30 minutes, 10 mW/cm²). b) Average film thickness versus target concentrations.

FIGS. 4A-4C. Detecting single base alterations using polymerization-based amplification. The 10 nM target solutions in buffer, containing either KRAS WT or KRAS Mutant (G→R), were separately added to an array with capture probe locations depicted in (a). The resultant digital camera images (b) demonstrates the visual discrimination between a single base variation (5′CTG→5′CTC) in KRAS codon 12. (c) Profilometry scans indicate film thicknesses of approximately 80 nm resultant of complimentary hybridization.

### DETAILED DESCRIPTION OF THE INVENTION

In order for molecular interaction between the target and the probe to identify the target, the molecular interaction between the target and the probe must be sufficiently specific. For hybridization, the selectivity is a measure of the specificity of the molecular recognition event. “Selectivity” or “hybridization selectivity” is the ratio of the amount of hybridization (i.e., number of second nucleic acids hybridized) of fully complementary hybrids to partially complementary hybrids, based on the relative thermodynamic stability of the two complexes. For the purpose of this definition it is presumed that this ratio is reflected as an ensemble average of individual molecular binding events. Selectivity is typically expressed as the ratio of the amount of hybridization of fully complementary hybrids to hybrids having one base pair mismatches in sequence. Selectivity is a function of many variables, including, but not limited to: temperature, ionic strength, pH, immobilization density, nucleic acid length, the chemical nature of the substrate surface and the presence of polyelectrolytes and/or other oligomers immobilized on the substrate or otherwise associated with the immobilised film. For hybridization, the extent to which the sequences of the target and probe molecules are complementary influences whether hybridization occurs.

In an embodiment, either the target or the probe is a nucleic acid. In an embodiment, both the target and the probe comprise a single stranded nucleic acid. In an embodiment, the probe comprises an oligonucleotide, a relatively short chain of single-stranded DNA or RNA. “Nucleic acid” includes DNA and RNA, whether single or double stranded. The term is also intended to include a strand that is a mixture of nucleic acids and nucleic acid analogs and/or nucleotide analogs, or that is made entirely of nucleic acid analogs and/or nucleotide analogs and that may be conjugated to a linker molecule. “Nucleic acid analogue” refers to modified nucleic acids or species unrelated to nucleic acids that are capable of providing selective binding to nucleic acids or other nucleic acid analogues. As used herein, the term “nucleotide analogues” includes nucleic acids where the internucleotide phosphodiester bond of DNA or RNA is modified to enhance bio-stability of the oligomer and “tune” the selectivity/specificity for target molecules (Uhlmann, et al., (1990), Angew. Chem. Int. Ed. Eng., 90: 543; Goodchild, (1990), J. Bioconjugate Chem., I: 165; Englisch et al., (1991), Angew, Chem. Int. Ed. Eng., 30: 613). Such modifications may include and are not limited to phosphorothioates, phosphorodithioates, phosphotriesters, phosphoramidates or methylphosphonates. The 2′-O-methyl, allyl and 2′-deoxy-2′-fluoro RNA analogs, when incorporated into an oligomer show increased biostability and stabilization of the RNA/DNA duplex (Lesnik et al., (1993), Biochemistry, 32: 7832). As used herein, the term “nucleic acid analogues” also include alpha anomers (α-DNA), L-DNA (mirror image DNA), 2′-5′ linked RNA, branched DNA/RNA or chimeras of natural DNA or RNA and the above-modified nucleic acids. For the purposes of the present invention, any nucleic acid containing a “nucleotide analogue” shall be considered as a nucleic acid analogue. Backbone replaced nucleic acid analogues can also be adapted to for use as immobilized selective moieties of the present invention. For purposes of the present invention, the peptide nucleic acids (PNAs) (Nielsen et al., (1993), Anti-Cancer Drug Design, 8: 53; Engels et al., (1992), Angew, Chem. Int. Ed. Eng., 31: 1008) and carbamate-bridged morpholino-type oligonucleotide analogs (Burger, D. R., (1993), J. Clinical Immunoassay, 16: 224; Uhlmann, et al., (1993), Methods in Molecular Biology, 20, “Protocols for Oligonucleotides and Analogs,” ed. Sudhir Agarwal, Humana Press, NJ, U.S.A., pp. 335-389) are also embraced by the term “nucleic acid analogues”. Both exhibit sequence-specific binding to DNA with the resulting duplexes being more thermally stable than the natural DNA/DNA duplex. Other backbone-replaced nucleic acids are well known to those skilled in the art and can also be used in the present invention (See e.g., Uhlmann et al., (1993), Methods in Molecular Biology, 20, “Protocols for Oligonucleotides and Analogs,” ed. Sudhir Agrawal, Humana Press, NJ, U.S.A., pp. 335). Single-stranded nucleic acids (including analogs) suitable for use in probes of the present invention provide a binding portion which is capable of polymerase-mediated, template-dependent primer extension.

More generally, the probe and/or target can be or can comprise an oligomer. “Oligomer” refers to a polymer that consists of two or more monomers that are not necessarily identical. Oligomers include, without limitation, nucleic acids (which include nucleic acid analogs as defined above), oligoelectrolytes, hydrocarbon based compounds, dendrimers, nucleic acid analogues, polypeptides, oligopeptides, polyethers, oligoethers any or all of which may be immobilized to a substrate. Oligomers can be immobilized to a substrate surface directly or via a linker molecule.

In an embodiment, the probe comprises DNA. The DNA may be genomic DNA or cloned DNA. The DNA may be complementary DNA (cDNA), in which case the target may be messenger RNA (mRNA). The DNA may also be an Expressed Sequence Tag (EST) or a Bacterial Artificial Chromosome (BAC). For use in hybridization microarrays, double-stranded probes are denatured prior to hybridization, effectively resulting in single-stranded probes.

In an embodiment, the probe comprises a single stranded nucleic acid which comprises a first region (binding or capture region) connected to a second region (spacer region). In an embodiment, a 5′ modifier of the spacer region of the probe allows its attachment to a solid substrate. Suitable modifiers are known to the art and include hydrazide or amino modifiers. In an embodiment, the probe is covalently bound to the substrate.

In an embodiment, a probe is selected for use with a target to be detected. A portion of the probe, the binding portion, is selected to have a sequence at least partially complementary to a portion of the target. This portion of the probe may also be termed the capture portion, since it is designed to capture a selected target. In an embodiment, perfectly complementary binding will occur between the binding portions of the probe and of the selected target. However, other functional probe designs need not involve perfectly complementary binding along the whole length of the binding regions. The binding portion of the probe may be sufficiently similar to another target to allow hybridization to occur. In another embodiment, for a target other than the selected target, the sequences of the target and probe binding portions may be sufficiently incompatible that no binding will occur.

In an embodiment, the length of the binding region of the probe (number of bases) is selected to be less than the length of the target nucleotide. In different embodiments, the length of the binding portion of the probe is from 15 to 30 bases or 15 to 40 bases. In an embodiment, the binding region of the probe is selected to be at least partially complementary to a corresponding binding region of the target. In different embodiments, at least 7 out of 10, 8 out of 10, 9 out of 10 or greater than 9 out of 10 bases of the binding region of the probe are complementary to the binding region of the target. In an embodiment, the complementarity of bases at or near the 3′ terminus of the probe is especially important, since mismatches in this region have a large effect on whether primer extension can occur. In an embodiment, the base at the 3′ terminus of the probe is complementary to the corresponding base in the target binding region. In an embodiment, the number of mismatches in a region 10 bases from the 3′ terminus of the probe is minimized (terminus plus 9 bases back). In an embodiment, at least 8 out of 10 (e.g. 80%), 9 out of 10 or greater than 9 out of 10 bases at or near the 3′ end of the binding region of the probe are complementary to the binding region of the target.

In an embodiment, the spacer region of the probe is selected so that the 3′ end of the target is not complementary to the corresponding spacer region. In particular, there is sufficient mismatch at the 3′ end of the target to prevent primer extension from this terminus. In an embodiment, the length of the spacer region is greater than the length of the target capping region. In an embodiment, the length of the spacer is from 12 to 25 bases.

In an embodiment, the target is a single stranded nucleic acid comprising a capture region, the capture being located intermediate to the 3′ and 5′ ends of the nucleic acid. In an embodiment, the 3′ end of the target is not complementary to the spacer region of the probe.

DNA microarrays are known to the art and commercially available. The general structure of a DNA microarray is a well defined array of spots on an optically flat surface, each of which contains a layer of relatively short strands of DNA. As referred to herein, microarrays have a spot size less than about 1.0 mm. In most hybridization experiments, 15-25 nucleotide sequences are the minimum oligonucleotide probe length (Schena, M., “Microarray Analysis, (2003) John Wiley & Sons, New Jersey, p. 8). The substrate is generally flat glass primed with an organosilane that contains an aldehyde functional group. The aldehyde groups facilitate covalent bond formation to biomolecules with free primary amines via Schiff base interactions. After reaction the chip is cured to form a very stable array ready for hybridization. In another aspect the biomolecules may be bound to the surface with an amine functionality, rather than an aldehyde functionality.

For probes bound to a substrate using aldehyde attachment chemistry, the substrate may be treated with an agent to reduce the remaining aldehydes prior to contacting the probe with the target. One suitable reducing agent is sodium borohydride NaBH₄. Such a treatment can decrease the amount of reaction between the monomer and the aldehyde coating on the glass, thus decreasing the amount of background signal during the detection step. The probe is contacted with a solution comprising the target under conditions effective to form a target-probe complex. The conditions effective to form a target-probe complex depend on the target and probe species. For ssDNA or RNA targets binding to ssDNA probes, suitable hybridization conditions have been described in the scientific literature. In an embodiment, it is sufficient to contact a solution comprising the target with the probe for about 2 hours at about 42° C. In other embodiment, the solution comprising the target may be contacted with the probe for longer amounts of time, such as 8 hours or more. In an embodiment, this solution also comprises an agent, such as a crowding agent, to limit nonspecific interactions. With reference to nucleic acid interactions, a crowding agent is an agent that interrupts nonspecific adsorption between nucleic acids that are not complementary. Formamide is one such agent to limit nonspecific interactions (Stahl, D. A., and R. Amann. 1991. Development and application of nucleic acid probes, p. 205-248. In E. Stackebrandt and M. Goodfellow (ed.), Nucleic acid techniques in bacterial systematics. John Wiley & Sons Ltd., Chichester, United Kingdom). Nonspecific interactions can also be limited by applying a blocking agent to the microarray prior to contacting the target with the probe. Suitable blocking agents are known to the art and include, but are not limited to bovine serum albumin (BSA), nonfat milk, and sodium borohydride. Detergents such as sodium lauroyl sarcosine or sodium dodecyl sulfate can also be added to aldehyde surface hybridization reactions to reduce background (Schena, M., “Microarray Analysis, (2003) John Wiley & Sons, New Jersey, p. 117). The target solution may also be contacted with the probe at higher temperatures in order to limit nonspecific interactions. Cross-hybridization can occur if the sequence complementarity between the target and the probe is greater than or equal to about 70% (Schena, M., “Microarray Analysis, (2003) John Wiley & Sons, New Jersey, p. 151). In different embodiments, the hybridization temperature is selected to favor hybrids having greater than or equal to 70%, 80%, 90% or 95% complementarity between binding region sequences.

In an embodiment, after the target is contacted with the probe, targets which have not formed target-probe complexes are removed. The unbound targets can be removed through rinsing. Water or an aqueous solution may be used for rinsing away unbound targets.

In DNA primer extension, DNA complementary to a template nucleic acid sequence can be synthesized by extending a primer able to hybridize to a portion of that template strand with a DNA polymerase. During the synthesis reaction, deoxynucleoside triphosphates (dNTPs) are typically incorporated to form a DNA fragment, although non-standard nucleotide triphosphates may also be incorporated during the extension reaction. The primer extension reaction employs a DNA polymerase enzyme that catalyzes the template-dependent incorporation of nucleotide(s) into the newly synthesized complementary strand. As used herein, the term “template nucleic acid molecule” refers to that strand of a nucleic acid from which a complementary nucleic acid strand is synthesized by a DNA polymerase, for example, in a primer extension reaction. The newly synthesized complementary strand is referred to herein as the “extension product”. The ability of a DNA polymerase to incorporate the correct deoxynucleotide is the basis for high fidelity DNA replication in vivo.

The duplex formed by hybridization of the target and probe is incubated in the presence of a DNA polymerase and a nucleotide triphosphate mixture. In an embodiment, the incubation processes of the invention involve contact of the duplex with a solution comprising the DNA polymerase and at least one nucleotide triphosphate at temperatures and polymerase and nucleotide triphosphate concentrations suitable for primer extension to occur. Typically, the primer extension reaction occurs at temperatures greater than room temperature.

A variety of DNA polymerases are known to the art. One classification scheme is based on structural similarity of a given polymerase to E. coli DNA polymerase I (Family A), II (Family B), or III (Family C). Examples of the Family A polymerases include, but are not limited to Klenow DNA polymerase, Thermus aquaticus DNA polymerase I (Taq polymerase) and bacteriophage T7 DNA polymerase. In an embodiment, the DNA polymerase is a Family A polymerase. In an embodiment, the DNA polymerase is a Klenow fragment which uses dsDNA as a primer (Pastinen, T. et al, 2000, 10:1031-1042; Erdogan, F. et al., 3001, 29(7), e36). In an embodiment, the DNA polymerase may have little or no proofreading ability, either inherently or through design. For example, forms of DNA polymerases are commercially available in which the exonuclease has been attenuated or eliminated (e.g. Klenow Fragment (3′→5′ exo-), Vent_{R}® (exo-) DNA Polymerase, both available from New England BioLabs, Ipswich, Mass.).

The duplex is also incubated in the presence of a nucleotide triphosphate mixture, the nucleotide triphosphate mixture comprising at least one biotin-labeled nucleotide. In an embodiment, the nucleotide triphosphate mixture comprises biotin-labeled and unlabeled deoxyribonucleotide triphosphate (dNTP) molecules. As is known in the art, dNTP molecules include dATP, dCTP, dGTP and dTTP. In an embodiment, the concentration of biotin-labeled dNTPs is sufficiently high to incorporate at least one biotin-labeled dNTP into the reaction product.

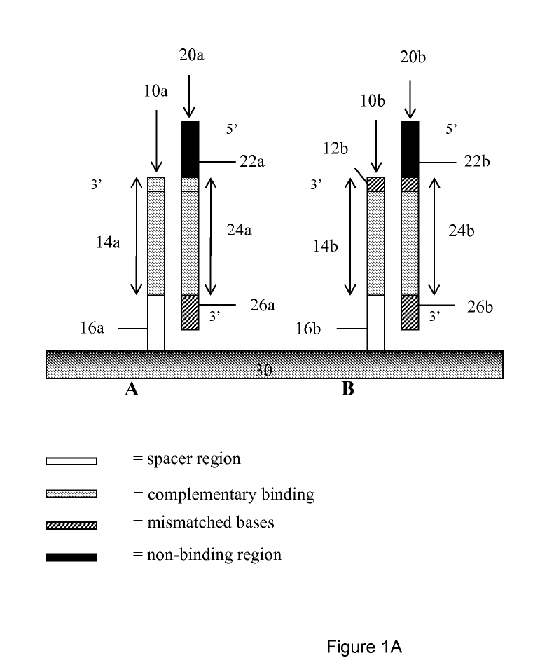

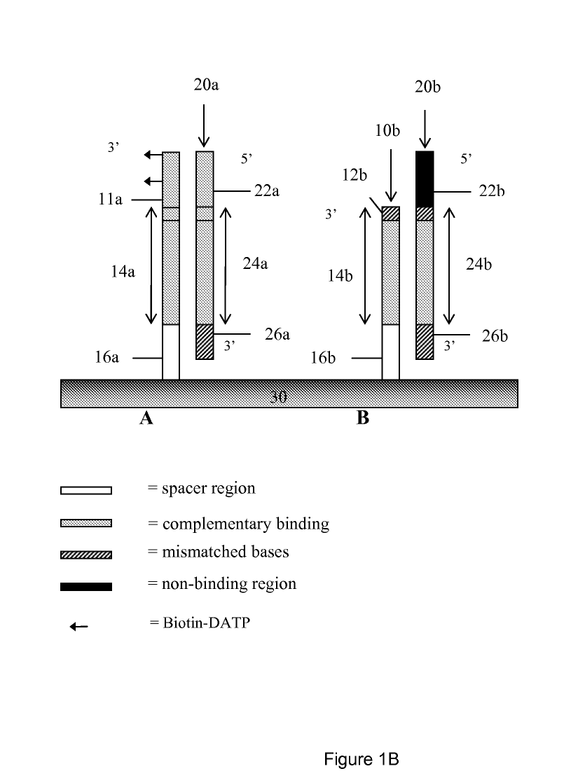

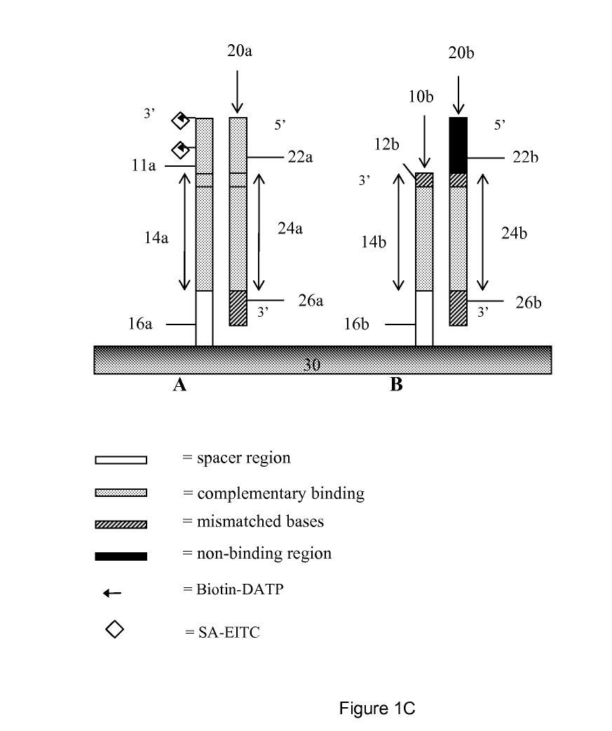

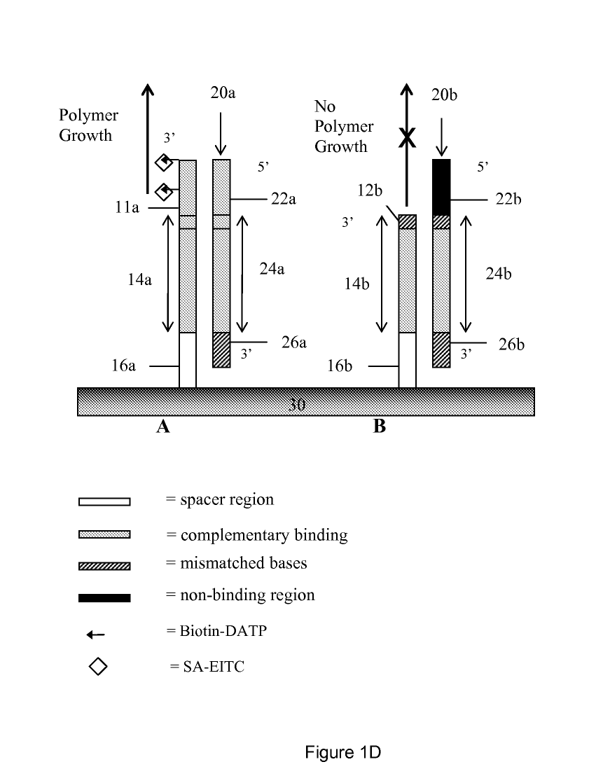

In an embodiment, the incubation takes place under conditions sufficient to permit extension of the 3′ terminus of the first region of the probe in a polymerase-mediated, template-dependent primer extension reaction. As shown in FIG. 1, the extension portion of the target acts as the template for formation of the extension product. At least one biotin-labeled nucleotide triphosphate is incorporated into the extension product. In an embodiment, a plurality of biotin-labeled nucleotide triphosphate molecules are incorporated into the extension product. In different embodiments, at least 2-10, 2-7, or 4-10 biotin molecules are incorporated into the extension product.

In an embodiment, the photoinitiator label comprises a photoinitiator and a biotin-binding protein. In an embodiment, the biotin-binding protein comprises avidin, strepavidin, or Neutravidin (a deglycosylated form of avidin). In an embodiment, the biotin-binding protein of the photoinitiator label binds to at least one biotin in the extension product attached to the probe. In another embodiment, the probe extension product is biotinylated, the photoinitiator label comprises a biotin moiety, and an intermediate biotin-binding moiety is used to connect the photoinitiator label to the target.

In an embodiment, photoinitiator molecules are attached to the biotin-binding protein. In an embodiment, a photoinitiator molecule can be attached to avidin or streptavidin by modification of avidin or streptavidin lysine residues. For photoinitiators having a carboxylic acid functional group, the carboxylic functional group of the photoinitiator can be coupled to the amine of the lysine residue in the presence of a coupling agent. The result is the formation of a peptide bond between the initiator and the protein. Suitable coupling agents are known to those skilled in the art and include, but are not limited to, EDC.

In another embodiment, a polymeric photoinitiator label is formed. Such a polymeric photoinitiator label can be formed from a polymer which can be coupled with both a photoinitiator and a molecular recognition group such as avidin or streptavidin. In another embodiment, the polymeric photoinitiator label can be formed from a polymer which can be coupled with both a photoinitiator and biotin. In an embodiment, the photoinitiator can be attached to the polymer by an ester linkage or by any other kind of linkage known to the art. In an embodiment, the avidin or streptavidin can be attached to the polymer by an amide linkage. In an embodiment, the polymer comprises an amine functional group or a carboxylic functional group. In an embodiment, the polymer comprises carboxylic acid groups and amide groups. In an embodiment, the polymer comprises a poly(acrylic acid-co-acrylamide) backbone.

In an embodiment, the polymer backbone of the macroinitiator comprises sufficient hydrophilic monomeric units that the macroinitiator is water soluble. In an embodiment, the hydrophilic monomeric units are selected from the group consisting of ethylene glycol, acrylate, acrylate derivatives such as acrylamide and hydroxyethylacrylate, and vinyl monomers such as 1-vinyl-2-pyrrolidinone. Without wishing to be bound by any particular belief, hydrophilic macroinitiator backbones are believed to limit nonspecific adsorption of the macroinitiator from aqueous solutions.

In an embodiment, a polymeric photoinitiator label can be formed from a polymer which comprises one part of a two-part photoinitiator system. The polymer can be coupled to a molecular recognition group such as avidin or streptavidin. When the combination of the polymer and the second part of the initiator system is exposed to the appropriate wavelength of light, the initiator system is capable of capable of initiating polymerization of a polymer precursor. In an embodiment, one part of the two-part photoinitiator system is a tertiary amine which is part of the polymeric photoinitiator label. The other part of the photoinitiator system can be camphorquinone. (CQ) This two-part system can be activated by light of approximately 469 nm. The tertiary amine can be incorporated into the polymer label by co-polymerizing acrylic acid with a monomer comprising the tertiary amine and an acrylate group.

In an embodiment, the polymeric photoinitiator comprises sufficient photoinitiators so that it may be regarded as a macroinitiator (having many initiators present on a single molecule). The number of initiator groups per molecule or chain may vary from one chain to another. In an embodiment, the use of a macroinitiator can increase the average initiator concentration by a factor of between about 10 to about 100. In other embodiments, the average number of initiators per polymer chain is from 50 to 100, from 80 to 180, from 120 to 160 or from 100 to 200. The number of molecular recognition groups may also vary from chain to chain. In an embodiment, the average number of molecular recognition groups is between one and three. Without wishing to be bound by any particular belief, it is believed that the incorporation of too many initiator groups can lead to nonspecific interaction between the macroinitiator and the array. The molecular weight of the backbone polymer is selected to be large enough to allow attachment of the appropriate number of initiator and molecular recognition groups. For a poly(acrylic acid-co-acrylamide) backbone, the molecular weight of the backbone is preferably greater than about 50,000.

U.S. Pat. No. 7,354,706 to Rowlen et al. and Sikes et al.(1), hereby incorporated by reference, describe experimental procedures for forming polymeric macroinitiators.

In an embodiment, the photoinitiator is activated by visible light. A number of photoinitiators are known to the art which can be activated by visible light to produce free radicals. In an embodiment, the photoinitiator is part of a two-part photoinitiator system, comprising a photoinitiator and a co-initiator. In an embodiment, the photoinitiator interacts with the co-initiator to generate free-radicals upon exposure to a source of visible light. In an embodiment, the photoinitiator is a photoreducible dye. Suitable co-initiators for visible light photoinitiators are known to those skilled in the art. In an embodiment, the visible light activated photoinitiator is water soluble and can be coupled to biotin or a biotin-binding protein.

In an embodiment, the photoinitiator label is selected so that the polymer produced through photopolymerization is bound to the extension product with sufficient strength that is not easily removed with rinsing (since the extension product is bound to a surface, the polymer will in turn be bound to the surface). In an embodiment, the photoinitiator molecule is selected so that the polymer formed is attached to the extension product through termination between surface stabilized radicals from the photoinitiator and bulk radicals present on the polymer chains. For example, it has been suggested that eosin radicals are responsible for strong attachment of polyethylene glycol (PEG) diacrylate gels onto substrate surfaces (20). In an embodiment, the photoinitiator is a xanthine dye. In one embodiment, the photoinitiator molecule is fluorescein or a fluorescein derivative. In an embodiment, the photoinitiator molecule is an eosin, a bromine derivative of fluorescein, or a derivative. In an embodiment, the photoinitiator molecule is 2′,4′,5′,7′-tetrabromofluorescein or a derivative. In an embodiment, the photoinitiator molecule is Rose Bengal or a derivative. In different embodiments, the photoinitiator is activated by wavelengths of light between 400 and 700 nm, between 450 and 600 nm, between 400 and 500 nm, or between 500 and 600 nm. Suitable co-initiators for fluorescein derivatives include, but are not limited to, amines such as methyl diethanol amine and tetraethanol amine.

In another embodiment, the photoinitiator is activated by ultraviolet (UV) light. In this embodiment, the monomer formulation is selected so to limit bulk polymerization by the wavelength of UV light. A number of UV-activated photoinitiators are known to the art. In an embodiment, the photoinitiator is a radical photoinitiator. In another embodiment, the photoinitiator is a cationic photoinitiator. In another embodiment, the photoinitiator comprises a carboxylic acid functional group. Commercially available photoinitiators, for example Irgacure 2959 (Ciba), can be modified to improve their water solubility.

Photoinitiators known to the art include azobisisobutyronitrile, peroxides, phenones, ethers, quinones, acids, formates. Cationic initiators include aryldiazonium, diaryliodonium, and triarylsulfonium salts. In an embodiment, the photoinitiator is selected from the group consisting of Rose Bengal (Aldrich), Darocur or Irgacure 2959 (2-hydroxy-1-(4-(hydroxyethoxy)phenyl)-2-methyl-1-propanone, D2959, Ciba-Geigy), Irgacure 651 (2,2-dimethoxy-2-phenylacetophenone, 1651, DMPA, Ciba-Geigy), Irgacure 184 (1-hydroxycyclohexyl phenyl ketone, 1184, Ciba-Geigy), Irgacure 907 (2-methyl-1-(4-(methylthio)phenyl)-2-(4-morpholinyl)-1-propanone, 1907, Ciba-Geigy), Camphorquinone (CQ, Aldrich), isopropyl thioxanthone (quantacure ITX, Great Lakes Fine Chemicals LTD., Cheshire, England). CQ is typically used in conjunction with an amine such as ethyl 4-N,N-dimethylaminobenzoate (4EDMAB, Aldrich) or triethanolamine (TEA, Aldrich) to initiate polymerization.

In an embodiment, the target-probe complex is contacted with the photoinitiator label under conditions effective to attach the photoinitiator label to the target-probe complex. In an embodiment, the target-probe complex is contacted with the photoinitiator label by contacting the target-probe complex with a photoinitiator label solution comprising the photoinitiator labels. In an embodiment, the solvent is aqueous and the photoinitiator label is water soluble. In an embodiment, the concentration of the photoinitiator label in the solution may be selected to limit nonspecific adsorption of the photoinitiator label to the surface of a substrate. In one embodiment where the photoinitiator label comprises eosin attached to streptavidin, the concentration of the photoinitiator label is 1 μg/mL or less.

In an embodiment, a blocking agent is used to limit nonspecific adsorption of avidin or other biotin-binding protein containing photoinitiator labels to the surface of an array. Suitable blocking agents are known to the art and include, but are not limited to, bovine serum albumin (BSA), nonfat milk, and sodium borohydride. PEG-based blocking agents which react with amine functionalities are also known to the art. The blocking agent may be applied to the substrate surface prior to contact of the photoinitiator label solution with the substrate, may be supplied in the photoinitiator label solution, or both. Denhardt's solution is a commercially available solution (Sigma-Aldrich) which contains BSA and can be included in the photoinitiator label solution.

After the photoinitiator label is attached to the target-probe complex, excess photoinitiator label is removed. In an embodiment, photoinitiator label not attached to the target-probe complex is removed to sufficiently reduce the any signal resulting from non-specific adsorption. The excess photoinitiator label may be removed by removal of the photoinitiator label solution. When the probe is attached to a solid substrate, the substrate may also be rinsed to remove the excess. The rinse may be a room temperature aqueous solution, such as a TNT solution (1M NaCl, 0.1M Tris, 0.1 wt % Tween 20). The rinse may also be at higher temperature, such through exposure to boiling water.

The photoinitiator-labeled complex is contacted with a polymer precursor or monomer solution. As used herein, a “polymer precursor” means a molecule or a portion thereof which can be polymerized to form a polymer or copolymer. Such precursors include monomers and oligomers. In this usage a monomer may itself contain a plurality of monomeric units. In an embodiment, the polymer precursor solution comprises at least one polymer precursor, co-initiator and a solvent. In an embodiment, the solvent comprises water and the polymer precursor is water soluble. The polymer precursor solution may also comprise other components, including molecules which serve to accelerate the polymerization reaction. In an embodiment, the concentration of the monomer components is selected to avoid excessive polymer film thickness, thereby facilitating quantitative determination of the number of molecular recognition events from the polymer film thickness. In an embodiment, the monomer solution is formulated to so that it does not unduly enhance propagation rates and or minimize termination rates, in contrast to formulations more suitable for encapsulation applications.

In an embodiment, the backbone of the monomer comprises sufficient hydrophilic monomeric units that the polymer precursor is water soluble. In an embodiment, the hydrophilic monomeric units are selected from the group consisting of ethylene glycol, acrylate, acrylate derivatives such as acrylamide and hydroxyethylacrylate, and vinyl monomers such as 1-vinyl-2-pyrrolidinone. In different embodiments, the molecular weight of the polymer is between 200 and 5000 or between 300 and 1000.

In an embodiment, the polymer precursor solution comprises a difunctional polymer precursor. In an embodiment the amount of difunctional polymer in the solution is from 5 up to 50 wt % (wt % as compared to the solution as a whole). In different embodiments, the amount of difunctional polymer precursor as compared to the total weight of polymer precursors in solution is at least 25 wt %, 50 wt %, 75 wt %, or 90% wt %. The inclusion of substantial amounts of difunctional monomer is believed to aid in the formation of greater amounts of polymer for a given polymerization time. For example, the presence of difunctional acrylate can yield pendant double bonds in propagating polymer chains that may crosslink with other propagating chains, thus suppressing chain termination rates and causing large amounts of high molecular weight polymer to be generated at the molecular recognition site.

In an embodiment, the solution comprises a difunctional polymer precursor with acrylate groups at each end. In an embodiment, the difunctional polymer precursor has a poly(ethylene glycol) (PEG) backbone and acrylate end groups. In an embodiment, the molecular weight of this difunctional PEG monomer is between 300 and 1000. In an embodiment, the weight percent of the difunctional PEG monomer in aqueous solution is from 5% to 50%. In an embodiment, an amine co-initiator is present in a concentration from 22.5 mM to 2250 mM. In an embodiment, the amine co-initiator is methyl diethanol amine. In an embodiment, an accelerant is present in a concentration from greater than zero to 250 nM. In an embodiment, the concentration of vinyl pyrrolidinone and MDEA are 30-40 mM and 200-250 mM, respectively. In an embodiment, the accelerator is 1-vinyl-2-pyrrolidinone.

In another embodiment, the solution comprises a mixture of a difunctional monomer with a vinyl group at each end and a monomer with a single vinyl group. In an embodiment, the polymer precursor solution comprises acrylamide and a bis-acrylamide crosslinker such as N,N-methylene-bis-acrylamide. As is known to the art, polymerization of these components forms polyacrylamide gel; the structure of the gel (average pore size) is dependent upon the total amount of acrylamide present and the relative amount of cross-linker. In an embodiment, the total amount of acrylamide and bisacrylamide is 40 wt % in aqueous solution and 5 mole % of the acrylamide is N,N-methylene-bis-acrylamide. In an embodiment, the amine co-initiator is methyl diethanol amine. In an embodiment, the accelerator is 1-vinyl-2-pyrrolidinone. These acrylamide solutions can be used produce thicker polymer coatings than some of the PEG solutions. In an embodiment, the concentration of vinyl pyrrolidinone and MDEA are 30-40 mM and 200-250 mM, respectively. This formulation is compatible with nitrocellulose-coated glass slides which are desirable for antibody array testing.

In an embodiment, the pH of the polymer precursor solution is greater than 7 and less than or equal to 9. In an embodiment, the pH of the polymer precursor solution is between 9 and 9. Since the pH of the solution can affect free radical formation, it is desirable to control the pH of the solution during the photopolymerization step.

In an embodiment, the amount of oxygen dissolved in the polymer precursor solution is minimized to minimize oxygen inhibition of the polymerization process. The amount of oxygen dissolved in the solution may be minimized by control of the atmosphere under which polymerization takes place, reducing the oxygen content of the polymer precursor solution by flowing a gas through it, or by a combination thereof. Suitable atmospheres and purge gases include, but are not limited to, argon and nitrogen. The amount of oxygen dissolved in the polymer precursor solution may also be controlled by the addition of oxygen inhibition agents.

The photoinitiator-labeled target-probe complex and polymer precursor solution are exposed to light, thereby forming a polymer. Photopolymerization occurs when polymer precursor solutions are exposed to light of sufficient power and of a wavelength capable of initiating polymerization. In an embodiment, the light source primarily provides light having a wavelength between 400 and 700 nm. In an embodiment, the intensity of the radiation is selected so that an appropriate dose of radiation can be delivered in less than about one-half hour.

In an embodiment, the polymer formed is a covalently crosslinked hydrogel. The term “hydrogel” refers to a class of polymeric materials which are extensively swollen in an aqueous medium, but which do not dissolve in water. Determination of polymer formation may be made with either swollen or dried gels. For accurate polymer film thickness measurements, the gels are typically dried.

The sensitivity of the detection methods of the invention can be measured in several ways. In an embodiment, a microarray dilution chip may be prepared having spots with differing amounts of a test molecule which is capable of binding with the photoinitiator label. The photoinitiator label is then attached to the test molecules. After photopolymerization, it may observed which spots on the chip result in a detectable amount of polymer formation. When the surface concentration of the test molecule of a given spot is known, polymer formation at the spot indicates detection of at least that concentration level of test molecule. When the size of the spot is known, the sensitivity can be determined in terms of the number of molecules required for detection. The sensitivity of the method determined by detection of a test molecule is expected to be related to the actual sensitivity of the method for detection of a target molecule, but may also be affected by factors such as labeling efficiency and hybridization efficiency. The assessed sensitivity of the method may depend on the sensitivity of method used to assess whether polymerization has occurred, with more sensitive polymerization assessment methods demonstrating greater sensitivity of the detection method. For example, when polymerization is assessed via optical observation the observed sensitivity may be lower then when polymerization is assessed via profilometry measurements. In an embodiment, the methods of the invention are capable of detecting a surface concentration of 0.4 attomoles (4×10⁻¹⁹ moles) when detection is assessed optically (by eye or with an optical microscope) and 0.1 attomoles when detection is assessed through profilometer measurements.

In an embodiment, the test molecule is a biotinylated oligonucleotide. Such a test molecule is useful when the photoinitiator label comprises a biotin-binding moiety. Methods for the determination of surface concentrations of oligonucleotides on bioarray surfaces are known to the art.

An amplification factor, which can be defined as the number of propagation reactions occurring per molecular recognition event, can be calculated from analysis of the thickness of the polymer films. The volume of the film may be divided by the density of the polymer to obtain the mass of polymer formed. Division by the molecular weight of the monomer/polymer precursor/repeat unit gives the number of monomers reacted into the polymer matrix. Dividing the number of monomers per micron squared by the number of molecular recognition events per micron squared gives the amplification factor. In an embodiment, the amplification factor of at least 106 or 107 is obtained for each molecular recognition event. If the amplification factor is determined across a given concentration range, the thickness of the film can be used as a measure of the number of molecular recognition events (in that concentration range).

In an embodiment, the number of molecular recognition events can be quantitatively determined by comparison of the observed film thickness to a calibration curve of film thickness versus probe concentration on a solid surface. The calibration curve is obtained for similar photopolymerization conditions (photoinitiator label, polymer precursor solution, light source, light intensity, and light exposure time). This calibration curve may be obtained by preparation of a dilution chip fabricated by spotting decreasing concentrations of probe molecules onto a substrate, then attaching photoinitiator labels to the probe molecules and photopolymerizing a monomer solution from the surface bound initiators according to the detection methods of the invention. The concentrations of the probe molecules can be determined for a similar dilution chip by attaching a fluorescent label to the probes and then characterizing the surface with fluorescent detection methods.

In another embodiment, the sensitivity of the detection methods of the invention may be measured by determining the minimum detectable concentration of target molecules for a particular set of amplification conditions. In an embodiment, the methods of the invention are capable of detecting target concentrations as low as 500 pM. In an embodiment, use of a macroinitiator photoinitiator label can enable detection of lower target concentrations such as concentrations as low as 1 pM, 5 pM, 10 pM, 10 pM, 50 pM or 100 pM.

Since a particular DNA polymerase may not equivalently distinguish all potential single base mismatches, some biotin-labeled extension product may form at mismatched hybrids as well as at perfectly matched hybrids. In the photoinitiator labeling step, some photoinitiator may be attached to these mismatched hybrids. In different embodiments, the photoinitiator concentration ratio of perfectly matched hybrids to mismatched hybrids is greater than or equal to 2:1, 5:1 or 10:1. In an embodiment, the polymer-amplified signal from the perfectly matched hybrids is visually distinguishable from the polymer-amplified signal from mismatched hybrids. In different embodiments, the polymer-amplified signal from mismatched hybrids is not detectable via visual observation or via profilometry. In another embodiment, the measured difference in polymer film thickness can be used to distinguish the polymer-amplified signal from perfectly matched hybrids from that of mismatched hybrids.

All references throughout this application, for example patent documents including issued or granted patents or equivalents; patent application publications; and non-patent literature documents or other source material; are hereby incorporated by reference herein in their entireties, as though individually incorporated by reference, to the extent each reference is at least partially not inconsistent with the disclosure in this application (for example, a reference that is partially inconsistent is incorporated by reference except for the partially inconsistent portion of the reference).

All patents and publications mentioned in the specification are indicative of the levels of skill of those skilled in the art to which the invention pertains. References cited herein are incorporated by reference herein in their entirety to indicate the state of the art, in some cases as of their filing date, and it is intended that this information can be employed herein, if needed, to exclude (for example, to disclaim) specific embodiments that are in the prior art. For example, when a compound is claimed, it should be understood that compounds known in the prior art, including certain compounds disclosed in the references disclosed herein (particularly in referenced patent documents), are not intended to be included in the claim.

Every formulation or combination of components described or exemplified can be used to practice the invention, unless otherwise stated. Specific names of compounds are intended to be exemplary, as it is known that one of ordinary skill in the art can name the same compounds differently. When a compound is described herein such that a particular isomer or enantiomer of the compound is not specified, for example, in a formula or in a chemical name, that description is intended to include each isomers and enantiomer of the compound described individual or in any combination. One of ordinary skill in the art will appreciate that methods, device elements, starting materials, and synthetic methods, other than those specifically exemplified can be employed in the practice of the invention without resort to undue experimentation. All art-known functional equivalents, of any such methods, device elements, starting materials, and synthetic methods, are intended to be included in this invention. Whenever a range is given in the specification, for example, a temperature range, a time range, or a composition range, all intermediate ranges and subranges, as well as all individual values included in the ranges given are intended to be included in the disclosure.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. Any recitation herein of the term “comprising”, particularly in a description of components of a composition or in a description of elements of a device, is understood to encompass those compositions and methods consisting essentially of and consisting of the recited components or elements. The invention illustratively described herein suitably may be practiced in the absence of any element or elements, limitation or limitations which is not specifically disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention.

In general the terms and phrases used herein have their art-recognized meaning, which can be found by reference to standard texts, journal references and contexts known to those skilled in the art. The preceding definitions are provided to clarify their specific use in the context of the invention.

### EXAMPLE

The polymerization-based assay described herein can provide visual, sensitive, and multiplexed detection of DNA hybridization from complex biological mixtures with a dynamic detection range. These assay capacities were demonstrated on simple glass microscope slides without the need for highly specialized biosensor surfaces. Further, the potential for dramatic improvements in signal specificity from allele-specific signal amplification is demonstrated through the visual discrimination between wild type (wt) and mutant alleles containing a single base mutation. Prominent genomic biomarkers targets commonly detectable from bodily fluids of cancer patients, including the KRAS proto-oncogene (15) and the p53 tumor suppressor gene (16) are used for demonstration.

Materials and Methods

Oligonucleotide Selection

The synthetic oligonucleotides from Integrated DNA Technologies (Coralville, Iowa) or Operon (Huntsville, Ala.) were ordered with HPLC or PAGE purification and were reconstituted in 10×TE buffer (pH 7.6) and stored at −20° C. upon receipt. The target sequences selected emulate human genomic regions with frequent mutations in either the KRAS gene or the p53 gene (Table 1). The capture sequence design included a 5′ hydrazide or amino modifier, a subsequent 21 base spacer region, and a 24 base binding region. The mutation site on the capture sequence occurred at the 3′ end to facilitate single base discrimination by the polymerase enzyme. The target sequence included a 3′ overhang of 6 bases lacking complementary base pairing to the capture that prevented PEX labeling of the target. Positive control capture probes included a 3′ tetra-ethyleneglycol (TEG) biotin modification

Microarray Fabrication

Amine functionalized glass slides from CEL Associates (Pearland, Tex.) were modified to aldehyde moieties using succinimidyl 4-formylbenzoate from SoluLink (San Diego, Calif.) according to published protocols (17). The in-house fabrication of the DNA microarrays occurred using a Biorad VerArray ChipWriter Pro (Hercules, Calif.) and a 375 μm diameter solid pin to deposit 8 μM of capture sequence in a spotting buffer containing 3×SSC and 0.05% SDS. The printed slides were incubated in a humid environment for 24 hours at ambient temperature to allow for complete coupling of capture probes to the surface. The further processing of the microarrays involved a NaBH₄ (Aldrich) reduction, a wash in 0.1% sodium dodecyl sulfate (Pierce Rockford, Ill.), a two minute immersion in boiling water, and a rinse in 2×SSC.

Hybridization and Primer Extension Labeling

A hybridization solution containing (0.2×SSC, 0.04×PBS OR [Na⁺]=50 mM, pH=7.0) was combined with the desired concentration of target sequence and subsequently presented to the DNA microarray and hybridized overnight at 45-55° C. A series of post-hybridization washes was performed including five minutes in 1×SSC, 0.1% SDS solution, two minutes in 0.1×SSC 0.1% SDS solution, and two minutes in 0.1×SSC solution. The slides were briefly rinsed with deionized water and dried. The DNA hybrids on the microarray were labeled using a primer extension reaction consisting of 500 U/mL of 3′-5′ exo-Klenow fragment (NEB Ipswich, Mass.), 20 μg/mL Extreme Thermostable Single Stranded Binding Protein (NEB), 50 μM each of dTTP, dCTP, dGTP (NEB), 125 mM biotin dATP (Invitrogen) in a buffer containing 10 mM Tris, 50 mM NaCl, 1 mM MgCl₂ and 1 mM dithiothreitol. Depending on the sensitivity and specificity parameters necessitated, variations in reaction temperatures, times, or inclusion of additional enzymes during PEX labeling were made. Under highly sensitive detection, standard PEX mix was added at 37° C. for 30 min. When a more rapid labeling was desired at the expense of sensitivity, standard PEX mix was added at 25° C. for 2 min. For highly specific, allele specific extension experiments, addition of 4000 mU/mL of apyrase enzyme (NEB) was added to reduce non-specific labeling by polymerase (12) (25° C. for 2 min). Arrays were rinsed for 2 minutes with deionized water.

Surface Initiated Photopolymerization

To achieve the desired bioselectivity of photoinitiation during amplification, biotinylated DNA sites from PEX labeling must be functionalized with initiator molecules. This outcome was achieved by functionalizing biotinylated DNA with a streptavidin-modified eosin isothiocyanate component (SA-EITC).

SA-EITC was synthesized as follows. The visible light photoinitiator Eosin-5-Isothiocyanate (Invitrogen) was functionalized directly onto external lysine residues of streptavidin through formation of a thiourea bond (Hermanson, G. T. Bioconjugate Techniques; Academic Press: San Diego, 1996) according to the reaction:

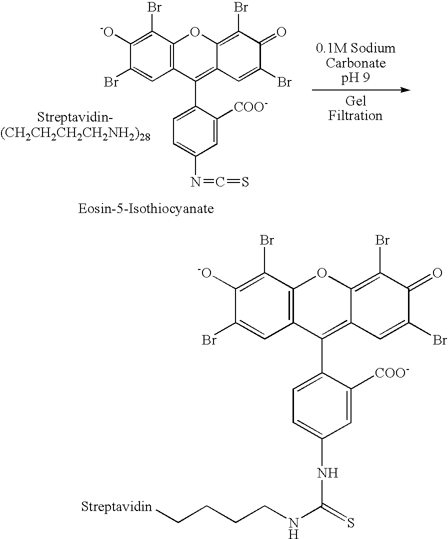

Streptavidin was dissolved in carbonate buffer (0.10 M NaCO₃, pH 9) at a concentration of 10 mg/mL. A 10 mg/mL solution of EITC in DMSO was prepared and immediately added to streptavidin at a volumetric ratio of 1:10. The solution was reacted for 8 hours at 4° C., then diluted to a streptavidin concentration of 1 mg/mL in 1×PBS and purified using gel filtration. The product was characterized with conventional UV-Vis spectroscopy, and the characteristic peak from EITC at 525 nm was compared to the characteristic protein peak at 280 nm according to equation 1:

$${n}_{EITC} / {n}_{SA} = \frac{A b {s}_{EITC , 525} / {ɛ}_{EITC , 525}}{( A b {s}_{SA , 280} - A b {s}_{EITC , 280} ) / {ɛ}_{SA , 280}} \qquad ( Equation 1 )$$

Measuring 0.2 mg/mL solution of the product diluted in 1×PBS buffer, an average photoinitiator to protein ratio of 2.3 was observed. The product was stored at 4° C. and protected from light exposure until further use.

Microarray functionalization of photoinitiator product was performed as follows. Spotted slides were blocked with 2 weight percent dry milk in ddH₂0 for 2 hours to prevent nonspecific adsorption of the photoinitiator product to the surface. Slides were then rinsed with water and contacted with 200 μL at 1 μg/mL of visible photoinitiator product in 1×PBS and 5× Denharts solution for 30 minutes. Slides functionalized with streptavidin-EITC were either placed in boiling water for two minutes or washed in TNT solution (1M NaCl, 0.1M Tris, 0.1 wt % Tween 20) to remove nonspecific protein adsorbed on the surface. Slides were rinsed in ddH₂O and allowed to dry

Functionalization of SA-EITC photoinitiator to appropriate sites on a microarray surface was verified with the use of an Agilent Technologies Microarray Scanner. The microarrays functionalized with SA-EITC were contacted with 700 μL of a precursor solution containing (polyethylene glycol (PEG) diacrylate 575, methyl diethanol amine, 1-vinyl-2-pyrrolidinone) (Sigma) (2). The microarray slide was secured in an incubation chamber (Whatman) and placed within an argon purged environment. An acticure light source was contacted onto the surface using either 500-695 nm, 10 mW/cm² intensity visible light for 40 minutes or 400-500 nm, 8 mW/cm² intensity for 20 minutes. The arrays were gently rinsed with water to remove unreacted monomer

Characterization of Amplified Microarray

Polymer films were stained using a hematoxylin solution (Mayer) to enhance their visibility. 1 mL of this solution was contacted with the amplified biochip for 1 hr followed by a 5 minute water wash. Visual detection of nucleic acid hybridization after amplification was confirmed either with the use of a digital camera or stereomicroscope to record a picture of an amplified biochip. Optical measurements on areas of an amplified chip after hematoxylin staining of polymer films was performed with the use a Leicia stereomicroscope that measured the absorbance of the array after illuminating the surface with a white-light fiber optic cable. Background readings used in signal to noise calculations were taken at areas outside the spot and across the array. Polymer growth was confirmed with the use of nanomechanical film thickness measurements using a Dektak 6M profilometer system, with a 12.5 μm diameter diamond stylus tip at a minimum stylus force of 1 mg to prevent polymer damage during measurement. This measurement yielded film thickness profiles of the polymer films if they were present at specific spots on the microarray surface.

Results and Discussion

Visual Detection of Nucleic Acid Hybridization

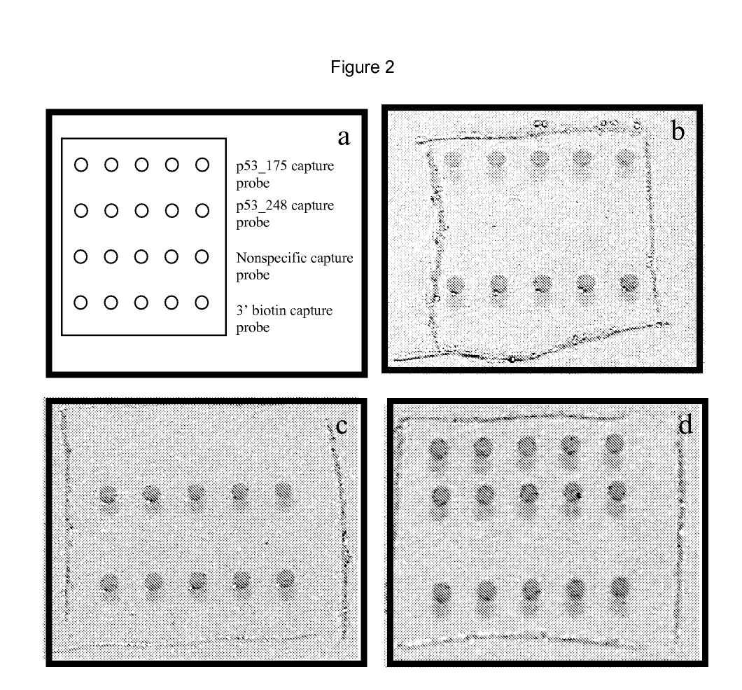

PEX labeling and signal amplification for multiplex DNA detection were investigated on microarray surfaces fabricated with parallel rows of spots containing p53-175, p53-248, and nonspecific capture probes, as well as a control row containing capture sequences with 3′ biotin modifications. After the complementary hybridization of nM concentrations of DNA targets, functionalization with photoinitiator, and the subsequent amplification, highly detectable, sub-micron thick polymer growth was obtained exclusively from spots that contain a minimal threshold initiator concentration (i.e., 4×10¹ initiators/μm²). As displayed in FIGS. 2a-d, the subsequent staining of the surface polymers with haematoxylin permits a visually discernible and specific characterization of one or more DNA targets. These highly visible and sequence-specific polymer films facilitate the elimination of complex instrumentation to detect oligonucleotide targets for rapid and high-throughput DNA sensing applications. FIG. 2a) Array layout of biochip used for detection of p53 targets. FIGS. 2b-d) Digital camera images of biochips for the detection of solutions of p53 target(s). FIG. 2b) 50 nM p53_{—}175 target in serum. FIG. 2c) 50 nM p53_{—}248 target in serum. FIG. 2d) 50 nM p53_{—}175 and 50 nM p53_{—}248 in serum. After hybridization, each array was labeled using PEX reactions (2 min., 25° C.), functionalized with SA-EITC, and amplified with polymerization-based amplification (30 min, 11 mW/cm²). PEG films where stained with hematoxylin to enhance their visibilities. Spots are 400 μm in diameter. Shadows appearing in images are due to the reflection of the spot onto the backside of the glass slide during the imaging process.

The covalent surface immobilization of molecular markers by PEX was unexpectedly crucial for achieving a high affinity between the surface and the grafted polymer films after the amplification step. Attempts at detecting DNA hybrids without covalent target immobilization resulted in unstable hydrogel polymer films (data not shown) and limited capacity for quantitative detection. Employing PEX eliminated delamination resulting from polymer swelling during subsequent washing steps. An additional finding from FIG. 2 is that this amplification system is also amenable to detection of nucleic acid markers from unpurified, complex mixtures of biomolecules. Here, DNA targets were spiked into serum and then detected without performing any additional purification steps. As a result, passive absorption of non-specific biomolecules onto the glass surface occurs during the initial hybridization step. The following assay steps—PEX labeling and amplification—still proceed from the surface with specificity despite the otherwise high densities of non-specific biomolecules. This observation is particularly significant during the amplification step, as significant non-specific initiation or inhibition of free radicals from serum components is not observed. This result is due in part to the low visible light absorptivity typical of biomolecules, rendering them unreactive to most visible light photochemical initiation pathways. This behavior highlights an advantage to using visible light for photoinitiation as opposed to ultraviolet radiation, where biomolecules typically contain higher molar extinction coefficients. This detection capability should also be readily translated to analysis of DNA from other complex biological mixtures common in molecular diagnostics, such as cell lysate.

Sensitivity and Dynamic Amplification

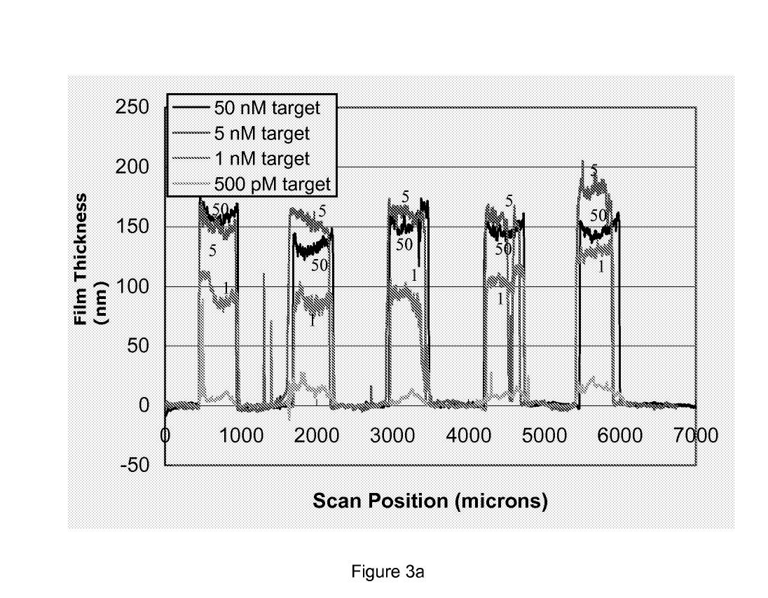

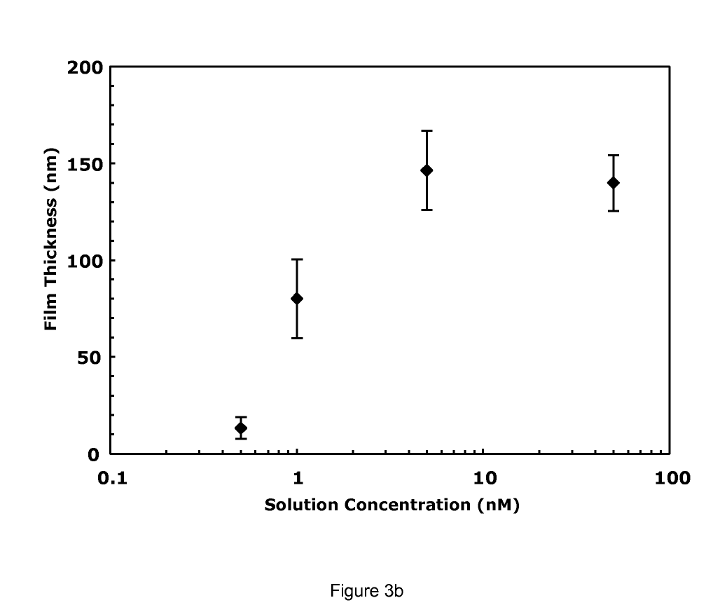

Evaluating the detection limits and the quantitative capacities of PBA for DNA hybridization is required to advance this amplification scheme from a simple biotin-SA detection system (1-3) to bio-recognition events valuable for biosensing applications. As previously reported, the amount of amplification as measured through polymer film thickness is a function of photoinitiator density (3). In hybridization reactions, the photoinitator surface density is dependent upon target solution concentration and the PEX photoinitiator labeling efficiency. This suggests the feasibility of a dynamic signal amplification and the presence of a lower limit of detection corresponding to the minimal surface-bound initiator density necessary for eliciting a detectable response, 4×10¹ initiators/μm² as revealed by fluorescence scanning methods. To investigate these parameters, the WT KRAS oligonucleotide target markers were diluted into hybridization buffer at specific concentrations, labeled with PEX for 30 minutes, and amplified at 10 mW/cm² for 30 minutes. FIG. 3 details the resulting film thicknesses elicited from each target concentration and the visual detection limits. Spots were visibly evident down to 500 μM target concentrations producing films of ˜20 nm, whereupon thinner films were not detectable by the unaided eye or from profilometry. Beyond 500 μM, the increasing target concentrations resulted in a dynamic increase of film thicknesses up to 5 nM as shown in FIGS. 3a and 3b. A saturation of film thickness occurred at concentrations above 5 nM likely associated with complete hybridization of the surface capture probes by the target, thus resulting in statistically similar photoinitiator concentrations after PEX labeling. The one order-of-magnitude dynamic range observed from FIG. 3b is readily extended towards higher-end target concentrations by employing SA:SA-EITC competitive binding techniques (3) or by using different capture probe concentrations (18,19). FIG. 3a) Profilometry height profiles across five replicate spots generated from decreasing amounts of target concentrations in buffer after PEX labeling, SA-EITC functionalization, and polymerization-based amplification (30 minutes, 10 mW/cm²). FIG. 3b) Average film thickness versus target concentrations.

The identification of DNA markers at sub-nanomolar concentration ranges using only commercialized reagents, simple bench-top chemistry, and glass microscope slides for visual detection is unique in bio-detection assays and has potential to provide significant advantages for point-of care (POC) implementation. Comparable DNA detection assays employing conventional microarray formats report lower detection limits (˜10 μM (20)), but typically employ fluorescent detection with expensive (˜$100,000) and sophisticated microarray scanners impractical for POC use. Similarly, novel SPR-based sensing methods report 10 μM sensitivity (21) and offer label-free detection, but simultaneously require cumbersome instrumentation in POC settings. While other approaches aimed at providing visual detection of nucleic acid targets including thin film biosensor technology (22), these assays often require use of specialized, optically treated biosensing surfaces. The current detection limits reported in this example would still require PCR target amplification schemes for detection in real samples. However, improved sensitivity is expected from the PBA method if higher numbers of eosin initiators are coupled to a hybridization event. This may be achieved through the synthesis of specialized macroinitiators containing higher ratios of eosin molecules to streptavidin proteins.

Visual Detection of Point Mutations

Since the unequivocal recognition of single base alterations is essential for many detection applications, applying PBA within this context further broadens its capabilities. Because the amplification scheme strictly depends upon the presence of initiators, preceding PEX discrimination scheme allows high specificity to be achieved. Here, the polymerization-based method exploited the Klenow (exo-3′-5′) polymerase to allocate initiator specifically to surface locations containing a perfectly matched allele of either wt or a (GAC:CGA) mutation in KRAS codon 12 targets without labeling hybrids containing a 3′ mismatch at the end of the primer capture probe. The targets were separately presented to fabricated microarrays that included both the wt and the mutant capture probes to permit detectable discrimination of variants. Apyrase enzyme was added in the PEX solution to facilitate allele-specific labeling through degradation of the biotin-dATP before the slower mismatch labeling by Klenow enzymes (12). The SA coupled initiator was then incubated on the surface followed by amplification.

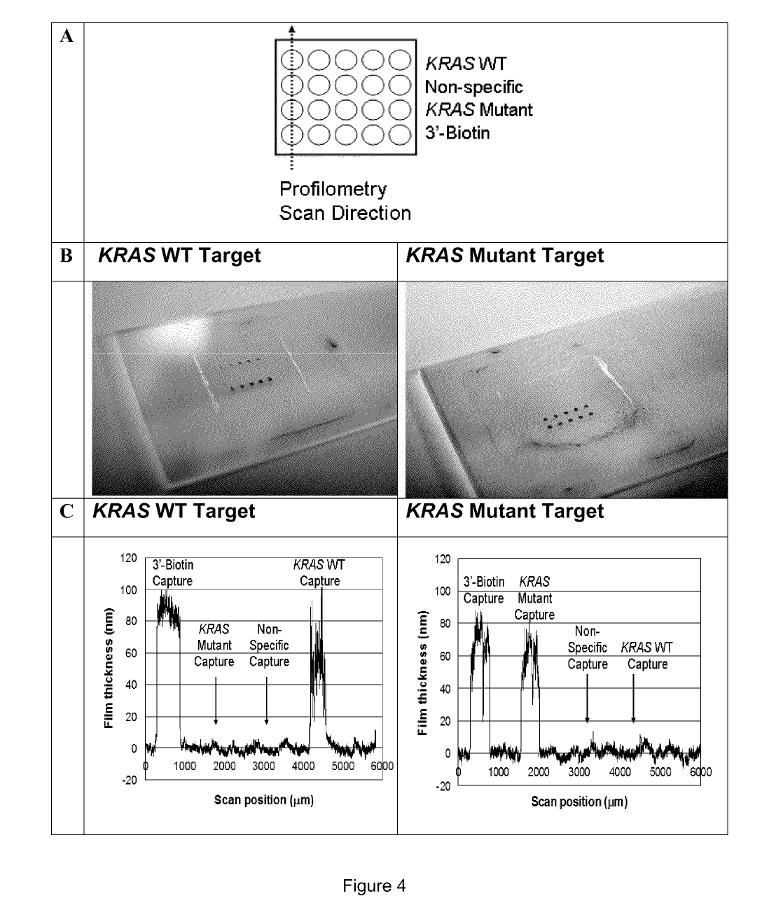

The photoinitiator densities presented in Table 2 demonstrate an allele-specific labeling reaction with a 10:1 initiator concentration ratio between perfectly matched sequences and mismatched sequences, representing the signal specificity achieved from allele-specific PEX only. This ratio is limited by nonspecific polymerase labeling that still occurs, as indicated by the higher initiator densities relative to the random, single stranded capture probes. These initiator densities, however, remain below the threshold level of detection (4×10¹ initiators/μm²) at which no detectable polymer growth can be detected. Conversely, initiator concentrations from perfectly matched hybrids are well above the threshold initiator level, allowing for highly detectable, nanoscale films to form on amplification. The post-amplification characterizations of each of the arrays through film thickness and optical microscope measurements shown in Table 2 reveal a strong positive signal without any detectable nonspecific signal, revealing a vast improvement in signal specificity that now approaches an infinite value. The simple digital camera images of these amplified biochips shown in FIGS. 4a-4c also highlight an advantageous aspect of this scheme for detecting point mutations, namely the ability for unmistakable positive responses evident from visual observations. FIGS. 4a-4c illustrate detection of single base alterations using polymerization-based amplification. The 10 nM target solutions in buffer, containing either KRAS WT or KRAS Mutant (G→R), were separately added to an array with capture probe locations depicted in FIG. 4(a). The resultant digital camera images in FIG. 4 (b) demonstrates the visual discrimination between a single base variation (5′CTG→5′CTC) in KRAS codon 12. FIG. 4(c) shows profilometry scans indicating film thicknesses of approximately 80 nm resultant of complimentary hybridization.

Numerous reports recognize the inconsistency in DNA polymerase to equivalently distinguish all potential single base mismatches. For example, polymerase easily extends C:A and most T mismatches (12,23). The dynamic behavior of the polymerization-based amplification method presents a significant and critical benefit for visually deciphering difficult mismatches such as G:T and C:A. In these cases, if nonspecific polymer growth is inevitable, the ratio between those polymerization signals generated from perfectly matched hybrids and from mismatched hybrids can be characterized such that only a significant increase from this background polymer growth indicates the presence of mutant DNA.

### CONCLUSIONS

This example describes approach for detecting nucleic acid hybridization events using a polymerization-based amplification scheme combined with PEX. The method described allow reliance upon the unaided eye for hybridization detection by monitoring polymer grown from a biofunctional photoinitiator that recognizes the hybrid and then initiates a polymerization reaction. The investigations reported here demonstrate the capacity for this technique to multiplex, detect markers from complex mixtures, and provide a dynamic detection range. Further, the discriminatory nature of polymerase enzymes, combined with the threshold nature of the amplification, allow for tremendous gains in signal specificity and allow for base-specific, visual identification of DNA targets. Although cancer biomarkers were implemented in these studies, the polymerization-based amplification scheme can be readily applied to a multitude of biodetection applications requiring information about nucleic acid sequences including detection of infectious disease or SNP genotyping for disease vulnerability. The aforementioned capabilities of this assay combined with its rapid, robust, and inexpensive nature make its implementation favorable, particularly in a clinic or POC setting where detection instrumentation is infeasible.

Finally, the recent development new POC technologies including electrochemical biosensors (24), potentiometric detection (25), and surface acoustic wave sensing systems (26), may benefit from the modular quality of the PBA system to potentially integrate with other surface biosensing schemes. With current sensitivities of these instruments in the nanomolar range, implementation of PBA systems into these devices may allow for considerable improvements in sensitivity.

TABLE 1
Oligonucleotide sequences utilized
with polymerization-based amplification

| Capture name | Sequence |
|---|---|
| KRAS codon 12 | 5′-CGGCAAACGGCATCAAACGGCATAAACTTGTGG |
| WT: | TAGTTGGAGCTG-3′ (SEQ ID NO:1) |
| KRAS codon 12 | 5′-CGGCAAACGGCATCAAACGGCATAAACTTGTGG |
| mutant (G→R): | TAGTTGGAGCTC-3′ (SEQ ID NO:2) |
| p53 codon 175 | 5′-CGGCAAACGGCATCAAACGGCCACATGACGGA |
| mutant (R→H): | GGTTGTGAGGCA-3′ (SEQ ID NO:3) |
| p53 codon 248 | 5′-CGGCAAACGGCATCAAACGGCTCCTGCATGGGC |
| mutant (R→Q): | GGCATGAACCA-3′ (SEQ ID NO:4) |
| Positive | 5′-CATCACACAACATCACACAACATCACGTATATA |
| control: | AAACGGAACGTAGAAGG-TEG-Biotin-3′ |
| | (SEQ ID NO:5 |
| Non-specific: | 5′-CATCACACAACATCACACAACATCACGTATATA |
| | AAACGGAACGTAGAAGG-3′ (SEQ ID NO:6) |
| Target name | Sequence |
| KRAS codon 12 | 5′-TTAGCTGTATCGTCAAGGCACTCTTGCCTACGC |
| WT: | CACCAGCTCCAACTACCACAAGTTTATATTCAG-3′ |
| | (SEQ ID NO:7) |
| KRAS codon 12 | 5′-AATTAGCTGTATCGTCAAGGCACTCTTGCCTAC |
| mutant (G→R): | GCCACGAGCTCCAACTACCACAAGTTTATATTCAG- |
| | 3′ (SEQ ID NO:8) |
| p53 codon 175 | 5′-ACCATCGCTATCTGAGCAGCGCTCATGGTGGGG |
| mutant (R→H): | GCAGTGCCTCACAACCTCCGTCATGTGCTGTGA-3′ |
| | (SEQ ID NO:9) |
| p53 codon 248 | 5′-GGAGTCTTCCAGTGTGATGATGGTGAGGATGGG |
| mutant (R→Q): | CCTCTGGTTCATGCCGCCCATGCAGGAACTGTT- |
| | 3′ (SEQ ID NO:10) |

TABLE 2

| | | Photoinitiator | Film | Signal to |
|---|---|---|---|---|
| | Capture | density | thickness | noise |
| Target | probe | (molecules/μm²) | (nm) | ratio (optical) |
| KRAS | 3′ Biotin | 510 +/− 30 | 80 +/− 10 | 7.4 |
| Wild | Nonspecific | 0 | <5 | 0.0 |
| Type | Mutant | 17 +/− 2 | <5 | 0.1 |
| | Wild type | 180 +/− 15 | 41 +/− 6 | 5.7 |
| KRAS | 3′ Biotin | 540 +/− 16 | 70 +/− 10 | 5.1 |
| Mutant | Nonspecific | 0 | <5 | 0.0 |
| | Mutant | 185 +/− 17 | 50 +/− 10 | 5.6 |
| | Wild type | 14 +/− 1 | <5 | 0.3 |

n, L. M. Johnson, R. Jenison, J. W. Birks, K. L. Rowlen, C. N. Bowman, Using polymeric materials to generate an amplified response to molecular recognition events, Nat. Mater. 7 (2008) 52-56.
(2) R. R. Hansen, H. D. Sikes, C. N. Bowman, Visual detection of labeled oligonucleotides using visible-light-polymerization-based amplification, Biomacromolecules 9 (2008) 355-362.
(3) R. R. Hansen, H. J. Avens, R. Shenoy, C. N. Bowman, Quantitative evaluation of oligonucleotide surface concentrations using polymerization-based amplification, Anal. Bioanal. Chem. 392 (2008) 167-175.
(4) D. Sidransky, Emerging molecular markers of cancer, Nat. Rev. Cancer 2 (2002) 210-219.
(5) J. D. Hoheisel, Microarray technology: beyond transcript profiling and genotype analysis, Nat. Rev. Genet. 7 (2006) 200-210.
(6) J. A. Ludwig, J. N. Weinstein, Biomarkers in cancer staging, prognosis and treatment selection, Nat. Rev. Cancer 5 (2005) 845-856.
(7) M. Weber, J. J. Davies, D. Wittig, E. J. Oakeley, M. Haase, W. L. Lam, D. Schubeler, Chromosome-wide and promoter-specific analyses identify sites of differential DNA methylation in normal and transformed human cells, Nat. Genet. 37 (2005) 853-862.
(8) G. Garcea, C. P. Neal, C. J. Pattenden, W. P. Steward, D. P. Berry, Molecular prognostic markers in pancreatic cancer: A systematic review, Eur. J. Cancer 41 (2005) 2213-2236.
(9) M. Wittek, M. Sturmer, H. W. Doerr, A. Berger, Molecular assays for monitoring HIV infection and antiretroviral therapy, Exp. Rev. Mol. Diag. 7 (2007) 237-246.
(10) R. M. Ratcliff, G. Chang, T. Kok, T. P. Sloots, Molecular diagnosis of medical viruses, Curr. Issues Mol. Biol. 9 (2007), 87-102.
(11) T. Pastinen, M. Raitio, K. Lindroos, P. Tainola, L. Peltonen, A. Syvanen, A system for specific, high-throughput genotyping by allele-specific primer extension on microarrays, Genome Res. 10 (2000) 1031-1042.
(12) D. O'Meara, A. Ahmadian, J. Odeberg, J. Lundeberg SNP typing by apyrase-mediated allele-specific primer extension on DNA microarrays, Nucleic Acids Res. (2002) e75.
(13) F. Erdogan, R. Kirchner, W. Mann, H. Ropers, U. A. Nuber, Detection of mitochondrial single nucleotide polymorphisms using a primer elongation reaction on oligonucleotide microarrays, Nucleic Acids Res. 29 (2001) e36.
(14) M. V. Myakishev, Y. Khripin, S. Hu, D. H. Hamer, High-throughput SNP genotyping by allele-specific PCR with universal energy-transfer-labeled primers, Genome Res. 11 (2001) 163-169.
(15) C. A. Ellis, G. Clark, The importance of being K-Ras, Cellular Signal. 12 (2000) 425-434.
(16) T. Soussi, C. Beroud, Significance of TP53 mutations in human cancer: A critical analysis of mutations at CpG dinucleotides, Hum. Mutat. 21 (2003) 192-200.
(17) X. Zhong, R. Reynolds, J. R. Kidd, K. K. Kidd, R. Jenison, R. A. Marlar, D. C. Ward, Single-nucleotide polymorphism genotyping on optical thin-film biosensor chips, Proc. Natl. Acad. Sci. 100 (2003) 11559-11564.
(18) W. Michel, T. Mai, T. Naiser, A. Oft, Optical study of DNA surface hybridization reveals DNA surface density as a key parameter for microarray hybridization kinetics, Biophys. J. 92 (2007) 999-1004.
(19) A. W. Peterson, R. J. Heaton, R. M. Georgiadis, The effect of surface probe density on DNA hybridization, Nucleic Acids Res. 29 (2001) 5163-5168.
(20) N. Zammatteo, L. Jeanmart, S. Hamels, S. Courtois, P. Louette, L. Hevesi, J. Remacle, Comparison between different strategies of covalent attachment of DNA to glass surfaces to build DNA microarrays, Anal. Biochem. 280 (2000) 143-150.
(21) D. K. Kim, K. Kerman, M. Saito, R. Sathuluri, T. Endo, S. Yamamura, Y. S. Kwon, E. Tamiyam, Label-free DNA biosensor based on localized surface plasmon resonance coupled with interferometry, Anal. Chem. 79 (2007) 1855-1864.
(22) R. Jenison, H. S. Yang, A. Haeberli, B. Polisky. Interference-based detection of nucleic acid targets on optically coated silicon. Nature Biotechnol. 19 (2001) 62-65.
(23) S. Creighton, M. M. Huang, H. Cai, N Arnheim, M. F. Goodman, Base mispair extension kinetics. Binding of avian myeloblastosis reverse transcriptase to matched and mismatched base pair termini, J. Biol. Chem. 267 (1992) 2633-2639.
(24) Y. Xiao, X. Qu, K. W. Plaxco, A. J. Heeger, Label-free electrochemical detection of DNA in blood serum via target-induced resolution of an electrode-bound DNA pseudoknot, J. Am. Chem. Soc. 129 (2007) 11896-11897.
(25) M. A. Lapierre, M. O'Keefe, B. J. Taft, S. O Kelly, Electrocatalytic detection of pathogenic DNA sequences and antibiotic resistance markers, Anal. Chem. 75 (2003) 6327-6333.
(26) T. M. A. Gronewold, A. Baumgartner, E. Quandt, M. Famulok, Discrimination of single mutations in cancer-related gene fragments with a surface acoustic wave sensor, Anal. Chem. 78 (2006) 4865-4871.

We claim:

1. A method for amplifying a molecular recognition interaction between a target and a probe, said method comprising the steps of:
a. providing a target single stranded nucleic acid (target), the target comprising in 3′ to 5′ order a first region (end cap region), a second region (binding region), and a third region (extension region);
b. providing a probe comprising a single stranded nucleic acid, said nucleic acid comprising in 3′ to 5′ order a first region (binding region) and a second region (spacer region), the 5′ end of the second region being bound to a solid substrate, wherein the first region of the target is of shorter length than and is not complementary to the second region of the probe, and the second region of the target is of equal length to and is at least partially complementary to the first region of the probe;
c. contacting the target with the probe under conditions effective to form a duplex between the probe and the target by hybridization of the first region of the probe to the second region of the target;
d. removing target not complexed with the probe;
e. incubating the duplex of step (c) in the presence of a DNA polymerase and a nucleotide triphosphate mixture, the nucleotide triphosphate mixture comprising at least one biotin-labeled nucleotide triphosphate, the incubation being under conditions sufficient to permit extension of the 3′ terminus of the first region of the probe in a polymerase-mediated, template-dependent primer extension reaction and incorporation of the biotin-labeled nucleotide triphosphate into an extension product, thereby forming a biotin-labeled target probe complex;
f. contacting the biotin-labeled target-probe complex with a photoinitiator label comprising a photoinitiator and a biotin binding protein under conditions effective to attach the photoinitiator label to the target-probe complex, thereby forming a photoinitiator-labeled target-probe complex;
g. removing photoinitiator label not attached to the target-probe complex;
h. contacting the photoinitiator-labeled target-probe complex with a polymer precursor solution;
i. exposing the photoinitiator-labeled target-probe complex and the polymer precursor solution to light, thereby forming a polymer; and
j. detecting the polymer formed in step (i), thereby detecting an amplified target-probe interaction.

2. The method of claim 1, wherein the target comprises single-stranded DNA ((ssDNA) or RNA and the probe comprises ssDNA.

3. The method of claim 1, wherein the sequence of the first region of the probe is at least 80% complementary to the sequence of the second region of the target.

4. The method of claim 1, wherein the base at the 3′ end of first region of the probe is complementary to the base at the 5′ end of the second region of the target.

5. The method of claim 1, wherein the nucleoside triphosphate mixture comprises a plurality of deoxyribonucleotide triphosphate (dNTP) molecules.

6. The method of claim 1, wherein the nucleoside triphosphate mixture comprises a plurality of biotin-labeled dNTP molecules

7. The method of claim 1, wherein the photoinitiator label comprises a plurality of photoinitiators attached to a biotin binding protein.

8. The method of claim 1, wherein the photoinitiator label comprises a plurality of photoinitiators and at least one biotin-binding protein attached to backbone of a second polymer.

9. The method of claim 6, wherein the average number of initiators attached to the backbone of the polymer is from 80 to 180.

10. The method of claim 1, wherein the polymer precursor solution is an aqueous solution and comprises a water soluble difunctional monomer.

11. The method of claim 1 wherein the wherein the photoinitiator is activated by visible light.

12. The method of claim 1 wherein the initiator is fluorescein or a fluorescein derivative.

13. A method for identifying a target, the method comprising the steps of:
a. providing a single stranded target nucleic acid (target), the target comprising in 3′ to 5′ order a first region (end cap region), a second region (binding region), and a third region (extension region);
b. providing a probe array comprising a plurality of different probes, wherein the probes are attached to a solid substrate at known locations, each probe comprising a single stranded nucleic acid, said nucleic acid comprising in 3′ to 5′ order a first region (binding region) and a second region (spacer region), the 5′ end of the second region being bound to a solid substrate, wherein the second region of the target is of equal length to and is at least partially complementary to the first region of at least one of the probes, and the first region of the target is of shorter length than and is not complementary to the second region of said probe, contacting the target with the probe array under conditions effective to form a to form a duplex between said probe and said target by hybridization of the first region of the probe to the second region of the target;
c. contacting the target with the probe under conditions effective to form a duplex between the probe and the target by hybridization of the first region of the probe to the second region of the target
d. removing target not complexed with the probe;
e. incubating the duplex of step (c) in the presence of a DNA polymerase and a nucleoside triphosphate mixture, the nucleoside triphosphate mixture comprising at least one biotin-labeled nucleoside triphosphate, the incubation being under conditions sufficient to permit extension of the 3′ terminus of the first region of said probe in a polymerase-mediated, template-dependent primer extension reaction, and incorporation of the biotin-labeled nucleoside triphosphate in an extension product, thereby forming a biotin-labeled target probe complex
f. contacting the biotin-labeled target-probe complex with a photoinitiator label comprising a photoinitiator and a biotin binding protein under conditions effective to attach the photoinitiator label to the target-probe complex, thereby forming a photoinitiator-labeled target-probe complex;
g. removing photoinitiator label not attached to the target-probe complex;
h. contacting the photoinitiator-labeled target-probe complex with a polymer precursor solution;
i. exposing the photoinitiator-labeled target-probe complex and the polymer precursor solution to light, thereby forming a polymer; and
j. detecting the polymer formed in step (i),
wherein the location of the polymer formed indicates said probe forms a target-probe complex with the target, thereby identifying the target.

14. The method of claim 13, wherein the target comprises single-stranded DNA ((ssDNA) or RNA and the probe comprises ssDNA.

15. The method of claim 13, wherein the sequence of the first region of the probe is at least 80% complementary to the sequence of the second region of the target.

16. The method of claim 13, wherein the base at the 3′ end of first region of the probe is complementary to the base at the 5′ end of the second region of the target.

17. The method of claim 13, wherein the nucleoside triphosphate mixture comprises a plurality of deoxyribonucleotide triphosphate (dNTP) molecules.

18. The method of claim 17, wherein the nucleoside triphosphate mixture comprises a plurality of biotin-labeled dNTP molecules.

19. The method of claim 13, wherein the photoinitiator label comprises a plurality of photoinitiators attached to a biotin binding protein.

20. The method of claim 13, wherein the photoinitiator label comprises a plurality of photoinitiators and at least one biotin-binding protein attached to backbone of a second polymer.

21. The method of claim 20, wherein the average number of initiators attached to the polymer backbone is from 80 to 180.

22. The method of claim 13, wherein the polymer precursor solution is an aqueous solution and comprises a water soluble difunctional monomer.

23. The method of claim 13 wherein the initiator is activated by visible light.

24. The method of claim 13 wherein the initiator is fluorescein or a fluorescein derivative.
